SEQUENCE LISTING (63 sequences)

SEQ ID NO: 1 - protein, 62 aa, Unknown
  Exemplary Protein Sequence.
LAVEVLFILDIVLNFRTTFVSKGQVVFAPKICLHYVTTWFLLDVIAALPFDLLHAKVNYFGA

SEQ ID NO: 2 - protein, 62 aa, Unknown
  Exemplary Protein Sequence.
LAVEVLFILDIVLNFRTTFVSKGQVVFAPKICLHYVTTWFLLDVIAALPFDLLHAKVNYVGA

SEQ ID NO: 3 - protein, 62 aa, Unknown; identical to SEQ ID NO: 2
  Exemplary Protein Sequence.

SEQ ID NO: 4 - protein, 62 aa, Unknown
  Exemplary Protein Sequence.
IAVEILFIIDIILNFRTTYVSKGQVIFEARICIHYVTTWFIIDLIAALPFDLLYANVTVSLV

SEQ ID NO: 5 - protein, 62 aa, Unknown
  Exemplary Protein Sequence.
IAVEILFIIDIILNFRTTYVSKGQVIFEARICTHYVTTWFIIDLIAALPFDLLYANVTVSLV

SEQ ID NO: 6 - protein, 62 aa, Unknown
  Exemplary Protein Sequence.
IAVEMLFILDIILNFRTTYVSQGQVVSAPRIGLHYLATWFFVDLIAALPFDLLYVNITTSLV

SEQ ID NO: 7 - protein, 62 aa, Unknown; identical to SEQ ID NO: 6
  Exemplary Protein Sequence.

SEQ ID NO: 8 - protein, 62 aa, Unknown
  Exemplary Protein Sequence.
IAVEMLFILDIILNFRTTYVSQGQVISAPRIGLHYLATWFFIDLIAALPFDLLYINITTSLV

SEQ ID NO: 9 - protein, 62 aa, Unknown; identical to SEQ ID NO: 8
  Exemplary Protein Sequence.

SEQ ID NO: 10 - protein, 67 aa, Unknown
  Exemplary Protein Sequence.
SIVDVIFFIDIVLNFHTTFVGPGGEVVSDPKVIMNYLKSWFIIDLLSCLPDVFNAFDRDEDGIGSLF

SEQ ID NO: 11 - protein, 67 aa, Unknown; identical to SEQ ID NO: 10
  Exemplary Protein Sequence.

SEQ ID NO: 12 - protein, 67 aa, Unknown
  Exemplary Protein Sequence.
SIVDVIFFADILLNFHTTFVGPGGEVVIEPSVIQNYFKSWFLIDLLSCLPDIFYMFKRDDERIGSLF

SEQ ID NO: 13 - protein, 67 aa, Unknown; identical to SEQ ID NO: 12
  Exemplary Protein Sequence.

SEQ ID NO: 14 - protein, 67 aa, Unknown
  Exemplary Protein Sequence.
SVVDVIFLVDIVLNFHTTFVGPGGEVISDPKLIMNYLKTWFVIDLLSCLPDIINAFENVDEGISSLF

SEQ ID NO: 15 - protein, 67 aa, Unknown; identical to SEQ ID NO: 14
  Exemplary Protein Sequence.

SEQ ID NO: 16 - protein, 80 aa, Unknown
  Exemplary Protein Sequence.
SIVDVIFLVDIVLNFHTTFVGPAGEVISDPKLIMNYLKTWFVIDLLSCLPDVINAFENVDEVSAFMGDPGKIGFADQIPP

SEQ ID NO: 17 - protein, 80 aa, Unknown; identical to SEQ ID NO: 16
  Exemplary Protein Sequence.

SEQ ID NO: 18 - protein, 80 aa, Unknown; identical to SEQ ID NO: 16
  Exemplary Protein Sequence.

SEQ ID NO: 19 - protein, 67 aa, Unknown
  Exemplary Protein Sequence.
SIVDVIFLVDIVLNFHTTFVGPAGEVISDPKLIMNYLKTWFVIDLLSCLPDVINAFENVDEGISSLF

SEQ ID NO: 20 - protein, 67 aa, Unknown; identical to SEQ ID NO: 19
  Exemplary Protein Sequence.

SEQ ID NO: 21 - protein, 97 aa, Unknown
  Exemplary Protein Sequence.
STKVTQELTELIEESMKEVKAYREELEAQLGPVTQETQARLSKELQAAQARVGADMEDVRNRLVLYRSEVHNMLGGTTEELRSRLASHLRKLRKRLL

SEQ ID NO: 22 - protein, 97 aa, Unknown
  Exemplary Protein Sequence.
STKVTQELTELIEESMKEVKAYREELEAQLGPVTQETQARLSKELQAAQARVGADMEDVRNRLVLYRSEVHNMLGGTTEELRSRLASHLRNVRKRLV

SEQ ID NO: 23 - protein, 96 aa, Unknown
  Exemplary Protein Sequence.
TQVIQELTVLMEETMKEVKAYREELEGQLAPMAQETQARVSKELQAAQARLGSDMEDLRNRLAQYRSEVQAMLGGSTEELRARMASHLRKLRKRLL

SEQ ID NO: 24 - protein, 95 aa, Unknown
  Exemplary Protein Sequence.
TQVIQELTALMEETMKEVKAYKEELEGQLGPMAQETQARVSKELQAAQARLGSDMEDLCGRLAQYRSEVQAMLGGSTEELRARMASHLRKPKRLL

SEQ ID NO: 25 - protein, 96 aa, Unknown
  Exemplary Protein Sequence.
TQVIQELTALMEETMKEVKAYKEELEGQLGPMAQETQARVSKELQAAQARLGSDMEDLRNRLAQYRSEVQAMLGGSTEELRARMASHLRKLPKRLL

SEQ ID NO: 26 - protein, 96 aa, Unknown
  Exemplary Protein Sequence.
SQVTQELTVLMEETMKEVKAYKAELEEQLGPMKEETQARLSKELQAAQARLGADMEDVRTRLAQYRSEVHTMLGGSTEELRARLSSHLRKMRKRLL

SEQ ID NO: 27 - protein, 96 aa, Unknown
  Exemplary Protein Sequence.
SQVTQELTALMDETMKELKAYKSELEEQLSPVAEETRARLSKELQAAQARLGADMEDVRSRLAQYRSEVQAMLGGSTDELRARLASHLRKLRKRLL

SEQ ID NO: 28 - protein, 96 aa, Unknown
  Exemplary Protein Sequence.
PQVTQELTTLMDETMKELKAYKSELEEQLSPVAEETRARLSKELQAAQARLGADMEDVRSRLVQYRSEVQAMLGGSTEELRARLASHLRKLRKRLL

SEQ ID NO: 29 - protein, 96 aa, Unknown; identical to SEQ ID NO: 28
  Exemplary Protein Sequence.

SEQ ID NO: 30 - protein, 96 aa, Unknown; identical to SEQ ID NO: 28
  Exemplary Protein Sequence.

SEQ ID NO: 31 - protein, 96 aa, Unknown
  Exemplary Protein Sequence.
SQVTQELTALMDETMKELKAYRSELEEQLTPVAEETRARLSKELQAAQARLGADMEDVRGRLVQYRGEVQAMLGGSTEELRARLASHLRKLRKRLL

SEQ ID NO: 32 - protein, 96 aa, Unknown
  Exemplary Protein Sequence.
SQVTQELRALMDETMKELKAYKSELEEQLTPVAEETRARLSKELQAAQARLGADMEDVRGRLVQYRGEVQAMLGGSTEELRVRLASHLRKLRKRLL

SEQ ID NO: 33 - protein, 96 aa, Unknown
  Exemplary Protein Sequence.
SQVTQELRALMDETMKELKAYKSELEEQLTPVAEETRARLSKELQAAQARLGADMEDVCGRLVQYRGEVQAMLGGSTEELRVRLASHLRKLRKRLL

SEQ ID NO: 34 - protein, 96 aa, Unknown
  Exemplary Protein Sequence.
SQVTQELRALMDETMKELKAYKSELEEQLTPVAEETRARLSKELQTAQARLGADMEDVCGRLVQYRGEVQAMLGGSTEELRVRLASHLRKLRKRLL

SEQ ID NO: 35 - protein, 96 aa, Unknown
  Exemplary Protein Sequence.
SQVTQELTALMDETMKELKAYKSELEEQLTPVAEETRARLSKELQAAQARLGADMEDVRGRLVQYRGEVQAMLGGSTEELRARLASHLRKLRKRLL

SEQ ID NO: 36 - protein, 96 aa, Unknown; identical to SEQ ID NO: 35
  Exemplary Protein Sequence.

SEQ ID NO: 37 - protein, 96 aa, Unknown
  Exemplary Protein Sequence.
SQVTQELTALMDETMKELKAYKSELEEQLTPVAEETRARLSKELQAAQARLGADMEDVRGRLAQYRGEVQAMLGGSTEELRARLASHLRKLRKRLL

SEQ ID NO: 38 - protein, 107 aa, Unknown
  Exemplary Protein Sequence.
TDVTQQLNTLFQDKLGNINTYADDLQNKLVPFAVQLSGHLTKETERVREEIQKELEDLRANMMPHANKVSGMFGDNVQKLQEHLRPYATDLQAQINAQTQDMKRQLT

SEQ ID NO: 39 - protein, 107 aa, Unknown
  Exemplary Protein Sequence.
TDVTQQLNTLFQDKLGNINTYADDLQNKLVPFAVQLSGHLTKETERVREEIQKELEDLRANMMPHANKVSQMFGDNVQKLQEHLRPYATDLQAQINAQTQDMKRQLT

SEQ ID NO: 40 - protein, 107 aa, Unknown
  Exemplary Protein Sequence.
TDVTQQLSTLFKDKLGDASTYADGVHNKLVPFVVQLSGHLAKETERVKEEIKKELEDLRDRMMPHANKVTQTFGENMQKLQEHLKPYAVDLQDQINTQTQEMKLQLT

SEQ ID NO: 41 - protein, 107 aa, Unknown
  Exemplary Protein Sequence.
TDVTQQLSTLFQDKLGDASTYADGVHNKLVPFVVQLSGHLAKETERVKEEIKKELEDLRDRMMPHANKVTQTFGENMQKLQEHLKPYAVDLQDQINTQTQEMKLQLT

SEQ ID NO: 42 - protein, 107 aa, Unknown
  Exemplary Protein Sequence.
TDVTQQLSTLFQDKLGDASTYADGVHNKLVPFVVQLSGHLAQETERVKEEIKKELEDQRDRMMPHANKVTQTFGENMQKLQEHLKPYAVDLQDQINTQTQEMKLQLT

SEQ ID NO: 43 - protein, 107 aa, Unknown; identical to SEQ ID NO: 41
  Exemplary Protein Sequence.

SEQ ID NO: 44 - protein, 107 aa, Unknown; identical to SEQ ID NO: 41
  Exemplary Protein Sequence.

SEQ ID NO: 45 - protein, 85 aa, Unknown
  Exemplary Protein Sequence.
AEITKQLNTLLQSNLQSANSYAEELQRRLVPFATELQAQLVQDSQRLKKQIQQELAELQAKLAPYADEVHQQIGTNIRELQAKLS

SEQ ID NO: 46 - protein, 85 aa, Unknown
  Exemplary Protein Sequence.
SELTQQLNTLFQDKLGEVNTYTEDLQKKLVPFATELHERLTKDSEKLKEEIRRELEELRARLLPHATEVSQKIGDNVRELQQRLG

SEQ ID NO: 47 - protein, 85 aa, Unknown
  Exemplary Protein Sequence.
SELTQQLNALFQDKLGEVNTYAGDLQKKLVPFATELHERLAKDSKKLKEEIRKELEEVRARLLPHANEVSQKIGENVRELQQRLE

SEQ ID NO: 48 - protein, 85 aa, Unknown
  Exemplary Protein Sequence.
SELTQQLNALFQDKLGEVNTYAGDLQKKLVPFATELHERLAKDSEKLKEEIRKELEEVRARLLPHANEVSQKIGENVRELQQRLE

SEQ ID NO: 49 - protein, 85 aa, Unknown
  Exemplary Protein Sequence.
SELTQQLNALFQDKLGEVNTYAGDLQKKLVPFATELHERLAKDSEKLKEEIGKELEELRARLLPHANEVSQKIGDNLRELQQRLE

SEQ ID NO: 50 - protein, 9 aa, Unknown
  Exemplary Protein Sequence.
QPSLFVRXA

SEQ ID NO: 51 - protein, 9 aa, Unknown
  Exemplary Protein Sequence.
QPSVFIRTR

SEQ ID NO: 52 - protein, 9 aa, Unknown
  Exemplary Protein Sequence.
QPSLFVRTF

SEQ ID NO: 53 - protein, 9 aa, Unknown
  Exemplary Protein Sequence.
RPSVYVREQ

SEQ ID NO: 54 - protein, 9 aa, Unknown
  Exemplary Protein Sequence.
RPSAYIREM

SEQ ID NO: 55 - protein, 9 aa, Unknown
  Exemplary Protein Sequence.
RPSIFVRES

SEQ ID NO: 56 - protein, 9 aa, Unknown
  Exemplary Protein Sequence.
RPSLFVREW

SEQ ID NO: 57 - protein, 9 aa, Unknown
  Exemplary Protein Sequence.
RGSLFIWKA

SEQ ID NO: 58 - protein, 9 aa, Unknown
  Exemplary Protein Sequence.
RGVVFIWKR

SEQ ID NO: 59 - protein, 9 aa, Unknown
  Exemplary Protein Sequence.
RGPLFVWKF

SEQ ID NO: 60 - protein, 9 aa, Unknown
  Exemplary Protein Sequence.
RWKVXVWDQ

SEQ ID NO: 61 - protein, 9 aa, Unknown
  Exemplary Protein Sequence.
RWYAYIWDM

SEQ ID NO: 62 - protein, 9 aa, Unknown
  Exemplary Protein Sequence.
RWEIFYWDS

SEQ ID NO: 63 - protein, 9 aa, Unknown
  Exemplary Protein Sequence.
RWILFVWDW

(19) United States
(12) Patent Application Publication
(10) Pub. No.: US 2005/0149269 A1
(43) Pub. Date: Jul. 7, 2005

(54) Browsable database for biological use

(76) Inventors: Paul D. Thomas, San Francisco, CA (US); Anish Kejariwal, San Francisco, CA (US); Michael J. Champbell, San Francisco, CA (US); Huaiyu Mi, Palo Alto, CA (US); Karen Diemer, Albany, CA (US); Nan Guo, San Lorenzo, CA (US); Istvan Ladunga, Foster City, CA (US); Betty Lazareva, Sunnyvale, CA (US); Anushya Muruganujan, Campbell, CA (US); Steven Rabkin, San Francisco, CA (US); Jody Vandergriff, San Francisco, CA (US); Olivier Doremieux, San Francisco, CA (US)

Correspondence Address: HARNESS, DICKEY & PIERCE, P.L.C., P.O. BOX 828, BLOOMFIELD HILLS, MI, 48303, US

(21) Appl. No.: 10/731,875

(22) Filed: Dec. 9, 2003

Related U.S. Application Data
(60) Provisional application No. 60/431,879, filed on Dec. 9, 2002.

(51) Int. Cl. G06F 19/00; G01N 33/48; G01N 33/50

(52) U.S. Cl. 702/19; 702/20

(57) ABSTRACT

The browsable database can allow for high-throughput analysis of protein sequences. One helpful feature may be a simplified ontology of protein function, which allows browsing of the database by biological functions. Biologist curators may have associated the ontology terms with Hidden Markov Models (HMMs), rather than individual sequences, so that they can be applied to additional sequences. To ensure accurate functional classification, HMMs may be constructed not only for families, but for curator-defined subfamilies, whenever family members have divergent functions or nomenclature. Multiple sequence alignments and phylogenetic trees, including curator-assigned information, can be available for each family. Various versions of the browsable database may include training sequences from all organisms in the GenBank non-redundant protein database, and the HMMs can be used to classify gene products across the entire genomes of human, and Drosophila melanogaster.

### CROSS-REFERENCE TO RELATED APPLICATIONS

This application claims the benefit of U.S. Provisional Application No. 60/431,879, filed on Dec. 9, 2002. The disclosure of the above application is incorporated herein by reference in its entirety.

### FIELD

The application generally relates to index and retrieval systems and methods applied to biological information, and particularly relates to family- and sub-family related libraries of functional indices providing access to multiple protein sequence alignments and philogenetic trees.

### BACKGROUND AND SUMMARY

The function of a protein generally correlates quite well with its evolutionary history. Large scale sequencing efforts (both genomic and mRNA-derived) have generated a great deal of protein sequence information. At the current time, most known (or inferred) protein sequences have not been assayed experimentally for function. However, because the evolutionary history of a protein family can be estimated using protein sequence information, the function of uncharacterized proteins can often be inferred based on sequence similarities (i.e. shared evolutionary history) with proteins that have been experimentally characterized. This is the basis for the great utility (and popularity) of pairwise sequence comparison algorithms such as BLAST. It is also the basis for more advanced sequence comparison algorithms, like PSI-BLAST and HMMs (Hidden Markov Models) that take advantage of the large numbers of protein sequences to construct statistical models of related proteins.

The problem the browsable database solves, on the scale of up to hundreds of thousands of proteins, is the following: how can sequence similarity be turned into a function prediction? The answer is: “it depends.” Experts in using BLAST and other pairwise sequence similarity searches sometimes forget just how much interpretation is required to take a query protein, get a list of information about related proteins and make a function prediction for the query. The most desirable pieces of information are generally:
How closely related is my protein to proteins of known (or inferred) function?
What are the annotated function(s) of the related proteins, and how reliable was the information underlying that annotation?
How consistent are the annotated functions of related proteins?
Does the region of similarity extend over the entire protein, or just part, and if just a part, is similarity over that region alone enough to infer function?
How reliable are the sequences themselves—could either my query or any related proteins be fragments, chimeras or contain frameshift or sequencing errors?
How specific is the function prediction one can make—for example if the most closely related protein is a serotonin receptor, is the relationship close enough to predict that the query is a serotonin receptor, or just that it is a seven-transmembrane protein of the rhodopsin class, or somewhere in-between?
   Because answers to these questions vary on a case-by-case basis, even experts can make these judgments more easily in protein families in which they have experience. Some of these questions require expertise in bioinformatics (how does the algorithm work, what does the statistical score mean, how was the annotation derived, what database does the information come from) while others require expertise in the biology (are two apparently different annotations actually synonyms, how specific are the functions or processes of interest, is this a family for which functional inference can be believed). Also, the increasing size of public databases often makes these inferences more difficult to make rather than easier, since search times are slower and lists of related proteins are larger. The browsable database can help interpret sequence similarity results, by doing as much of this interpretation as possible automatically.

The browsable database can allow for high-throughput analysis of protein sequences. One helpful feature is a simplified ontology of protein function, which allows browsing of the database by biological functions. Biologist curators may have associated the ontology terms with Hidden Markov Models (HMMs), rather than individual sequences, so that they can be applied to additional sequences. To ensure accurate functional classification, HMMs may be constructed not only for families, but for curator-defined subfamilies, whenever family members have divergent functions or nomenclature. Multiple sequence alignments and phylogenetic trees, including curator-assigned information, can be available for each family. Various versions of the browsable database may include training sequences from all organisms in the GenBank non-redundant protein database, and the HMMs can be used to classify gene products across the entire genomes of human, and Drosophila melanogaster.

There can be two aspects to making the interpretation correctly: bioinformatics and biology. In the browsable database, sophisticated bioinformatics analysis can provide the statistical framework for relationships between sequences, but expert biologists can make the correlation between sequence relationships and biological function. This is the an aspect in which browsable database differs from other “curated” databases such as SwissProt and Proteome on the one hand, and Pfam on the other. Proteome, for example, goes in-depth into the literature on individual proteins, and then summarizes this information and uses it to classify the protein into functional categories. This approach sees the protein as a stand-alone unit, and does not give guidelines on how to infer function of proteins that do not appear in the literature. One example involcves the paralogs Bone Morphogenetic Protein Receptor 1A and 1B. The browsable database can annotate both as having molecular functions serine/threonine protein kinase receptor and other cytokine receptor, and involved in biological processes skeletal development and receptor protein serine/threonine kinase signaling pathway. Proteome annotations in LocusLink classify BMPR1 as involved in the biological process: “TGFBETA RECEPTOR SIGNALLING PATHWAY” but no molecular function classification, while BMPR2 is classified as having molecular function: “TRANSMEMBRANE RECEPTOR PROTEIN SERINE/THREONINE KINASE” but no biological process classification. The browsable database classifications can be more consistent and complete because all proteins in a given family are curated at the same time and in their phylogenetic context.

Pfam, at the other end of the spectrum, is composed of statistical models that describe protein families. For many cases this information is not enough to specify the function of a protein. Any two proteins in a Pfam family are likely to be evolutionarily related, but may not share the same functions. One example of this is the Pfam model CNG_membrane—the model for the membrane-spanning segment of cyclic nucleotide-gated ion channels. This model also recognizes the EAG-related subfamily of voltage-gated (which are not cyclic nucleotide-gated) potassium channels. The browsable database subfamily models may make this distinction, while the browsable database family model can remain general. In this case, one subfamily of the database can be classified as ligand-gated ion channel while the other appears as voltage-gated ion channel. The family level model may be accurately classified as ion channel since all subfamilies share this more general function. When a new sequence is scored against the browsable database's HMM library, the inferred function depends on the relationship to classified sequences. In this case, if the best HMM score is to one of the subfamilies, then the new sequence can belong to that subfamily and can be classified as, e.g., a ligand-gated ion channel. If the best HMM score is to the database family model, it may mean the new sequence belongs in a novel subfamily and, in this case, can only be inferred to be an ion channel.

Another example is the sugar transporter Pfam model, which recognizes transporters for a variety of small molecules including inorganic phosphate. Again, the browsable database can capture this distinction with separate subfamily level models for different transporter specificities, as well as a general family level model for identifying new family members. The browsable database can explicitly map the relationship between these two different but correlated worlds: individual protein function and protein sequence similarity. The browsable database may include a library of HMMs at varying levels of specificity (built by a team of expert bioinformaticists) that can be directly related to protein function by a team of expert biologists.

Further areas of applicability will become apparent from the detailed description provided hereinafter. It should be understood that the detailed description and specific examples, while indicating various embodiments, are intended for purposes of illustration only and are not intended to limit the scope of the teachings thereof.

### BRIEF DESCRIPTION OF THE DRAWINGS

The teachings of the present application will become more fully understood from the detailed description and the accompanying drawings, wherein:

FIG. 1 is a block diagram illustrating a browsable database for biological use;

FIG. 2 is a screenshot illustrating a browsable database interface component permitting users to select to view a gene list; the transcript/protein list view illustrated in FIGS. 6-7 provides hyperlinks to protein data.

FIG. 3 is a screenshot illustrating a first portion of a gene list view of the browsable database;

FIG. 4 is a screenshot illustrating a second portion of a gene list view of the browsable database;

FIG. 5 is a screenshot illustrating a browsable database interface component permitting users to select to view a transcript/protein list;

FIG. 6 is a screenshot illustrating a first portion of a transcript/protein list view of the browsable database;

FIG. 7 is a screenshot illustrating a second portion of a transcript/protein list view of the browsable database;

FIG. 8 is a screenshot illustrating an interface component of the browsable database;

FIG. 9 is a screenshot illustrating an interface component of the browsable database;

FIG. 10 is a screenshot illustrating an interface component of the browsable database;

FIG. 11 is a screenshot illustrating an interface component of the browsable database;

FIG. 12 is a screenshot illustrating an interface component of the browsable database;

FIG. 13 is a screenshot illustrating an interface component of the browsable database;

FIG. 14 is a screenshot illustrating an interface component of the browsable database, including subfamily sequence numbers 6331348 (Seq. ID No. 1), 6754424 (Seq. ID No. 2), 8659557 (Seq. ID No. 3), 7514045 (Seq. ID No. 4), 3702618 (Seq. ID No. 5), 5804790 (Seq. ID No. 6), 7514051 (Seq. ID No. 7), 6912446 (Seq. ID No. 8), 12740409 (Seq. ID No. 9), 7293023 (Seq. ID No. 10), 399253 (Seq. ID No. 11), 7511533 (Seq. ID No. 12), 4731355 (Seq. ID No. 13), 12054892 (Seq. ID No. 14), 6625694 (Seq. ID No. 15), 6754422 (Seq. ID No. 16), 3790565 (Seq. ID No. 17), 2584733 (Seq. ID No. 18), 7514046 (Seq. ID No. 19), and 4504831 (Seq. ID No. 20);

FIG. 15 is a screenshot illustrating an interface component of the browsable database;

FIG. 16 is a screenshot illustrating an interface component of the browsable database;

FIG. 17 is a screenshot illustrating an interface component of the browsable database;

FIG. 18 is a screenshot illustrating an interface component of the browsable database;

FIG. 19 is a screenshot illustrating an interface component of the browsable database;

FIG. 20 is a screenshot illustrating an interface component of the browsable database, including subfamily sequence numbers 2388609 (Seq. ID No. 21), 461527 (Seq. ID No. 22), 7441520 (Seq. ID No. 23), 2119322 (Seq. ID No. 24), 416629 (Seq. ID No. 25), 10644783 (Seq. ID No. 26), 3913071 (Seq. ID No. 27), 114040 (Seq. ID No. 28), 114042 (Seq. ID No. 29), 3913070 (Seq. ID No. 30), 11066430 (Seq. ID No. 31), 178853 (Seq. ID No. 32), 4557325 (Seq. ID No. 33), 178849 (Seq. ID No. 34), 11066425 (Seq. ID No. 35), 11034803 (Seq. ID No. 36), 11066420 (Seq. ID No. 37), 114008 (Seq. ID No. 38), 8392909 (Seq. ID No. 39), 6680702 (Seq. ID No. 40), 191889 (Seq. ID No. 41), 12836356 (Seq. ID No. 42), 1703331 (Seq. ID No. 43), 109575 (Seq. ID No. 44), 3645997 (Seq. ID No. 45), 3913046 (Seq. ID No. 46), 2492913 (Seq. ID No. 47), 461521 (Seq. ID No. 48), and 71797 (Seq. ID No. 49);

FIG. 21 is a screenshot illustrating an interface component of the browsable database;

FIG. 22 is a screenshot illustrating an interface component of the browsable database;

FIG. 23 is a screenshot illustrating an interface component of the browsable database;

FIG. 24 is a screenshot illustrating an interface component of the browsable database;

FIG. 25 is a screenshot illustrating an interface component of the browsable database;

FIG. 26 is a screenshot illustrating an interface component of the browsable database;

FIG. 27 is a screenshot illustrating an interface component of the browsable database;

FIG. 28 is a screenshot illustrating an interface component of the browsable database;

FIG. 29 is a screenshot illustrating an interface component of the browsable database;

FIG. 30 is a screenshot illustrating an interface component of the browsable database;

FIG. 31 is a screenshot illustrating an interface component of the browsable database;

FIG. 32 is a screenshot illustrating an interface component of the browsable database;

FIG. 33 is a screenshot illustrating an interface component of the browsable database;

FIG. 34 is a screenshot illustrating an interface component of the browsable database;

FIG. 35 is a screenshot illustrating an interface component of the browsable database;

FIG. 36 is a screenshot illustrating an interface component of the browsable database;

FIG. 37 is a screenshot illustrating an interface component of the browsable database;

FIG. 38 is a screenshot illustrating an interface component of the browsable database;

FIG. 39 is a screenshot illustrating an interface component of the browsable database;

FIG. 40 is a screenshot illustrating an interface component of the browsable database;

FIG. 41 is a screenshot illustrating an interface component of the browsable database;

FIG. 42 is a screenshot illustrating an interface component of the browsable database;

FIG. 43 is a screenshot illustrating an interface component of the browsable database;

FIG. 44 is a screenshot illustrating an interface component of the browsable database;

FIG. 45 is a screenshot illustrating an interface component of the browsable database;

FIG. 46 is a screenshot illustrating an interface component of the browsable database;

FIG. 47 is a block diagram illustrating organization of sequences into families by multiple domains;

FIG. 48 is a flow diagram illustrating generation of statistical models for predefined families and subfamilies;

FIG. 49 is a flow diagram illustrating assignment of families and subfamilies to biological process and molecular function categories, including subfamily sequence numbers Seq. 1A (Seq. ID No. 50), Seq. 2A (Seq. ID No. 51), Seq. 3A (Seq. ID No. 52), Seq. 4A (Seq. ID No. 53), Seq. 5A (Seq. ID No. 54), Seq. 6A (Seq. ID No. 55), Seq. 7A (Seq. ID No. 56), Seq. 1B (Seq. ID No. 57), Seq. 2B (Seq. ID No. 58), Seq. 3B (Seq. ID No. 59), Seq. 4B (Seq. ID No. 60), Seq. 5B (Seq. ID No. 61), Seq. 6B (Seq. ID No. 62), and Seq. 7B (Seq. ID No. 63).

### DETAILED DESCRIPTION

The following description is merely exemplary in nature and is in no way intended to limit the teachings, their application, or uses.

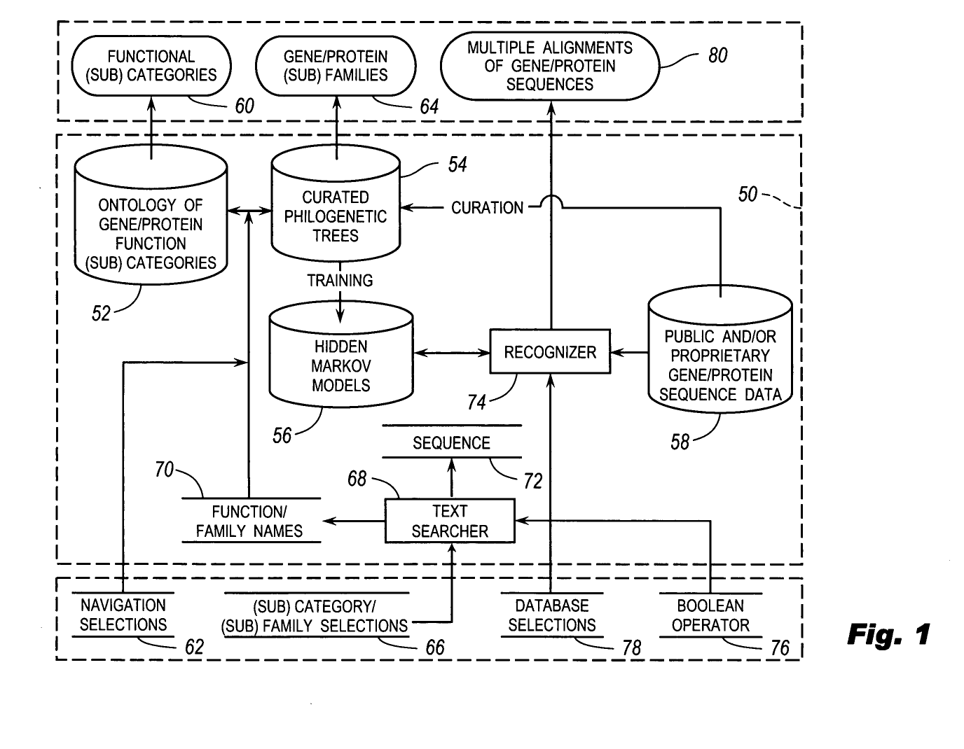

Referring to FIG. 1, the browsable database system 50 for biological use can include an ontology 52 of gene/protein function categories and subcategories. The categories may be related to curated philogenetic trees 54 of gene/protein sequence families and subfamilies. Curators may have divided families of sequences according to biological function and assigned them to appropriate categories and subcategories of ontology 52. Each family and subfamily of trees 54 may have an associated statistical model 56 trained on families and subfamilies of multiple sequences taken from sequence data 58 exhibiting the associated functions. Hidden Markov Models (HMMs) are one example of a statistical model that can be used.

Users interfacing with system 50 may view the ontology 52 at 60, and browse the ontology 52 by inputting navigation selections 62. Users may also view the families and subfamilies in the context of philogenetic trees 54 at 64, and browse the tree contents using navigation selections 62. Accordingly, users may select functional categories and subcategories, and gene/protein families and subfamilies by employing navigation selections 62. In some embodiments, selecting functional categories and subcategories may effectively accomplish selection of associated families and subfamilies. Accordingly, the statistical models 56 associated with the families and subfamilies of trees 54 may be equivalently associated with the functional categories and subcategories of ontology 52. It is envisioned that various embodiments may not include trees 54, but may instead include ontology 52 mapped directly to statistical models 56 trained on sequences exhibiting related functions. It should be readily understood that genes and proteins are substantially functionally equivalent to one another as well as substantially co-determinable via transcription. Thus, gene function may be used interchangeably with protein function in the present application. Gene sequence and protein sequence may similarly used interchangeably.

Users may also select functional categories, functional subcategories, functional families, and functional subfamilies using a text search. Accordingly, users input textual selections 66 to text searcher 68 in the form of names of functions and/or families 70 and/or sequences 72. Names 70 are matched to contents of ontology 52 and trees 54 to accomplish the selection. Sequences 72 are passed to recognizer 74, which selects tree 74 and ontology 52 contents related to statistical models 56 that score well against sequence 72.

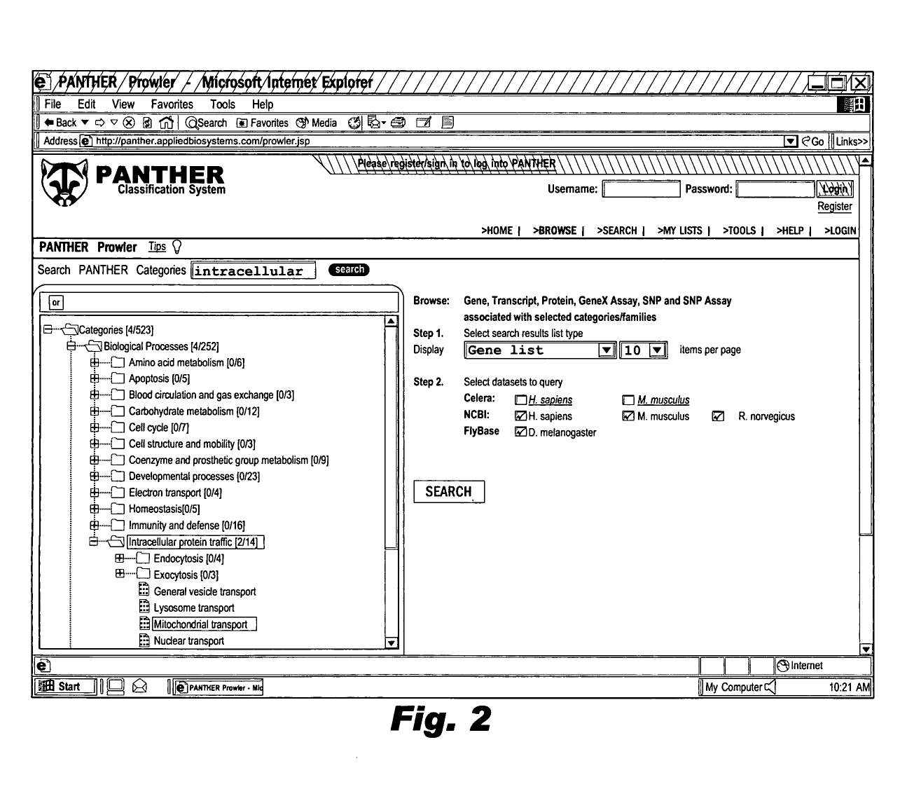

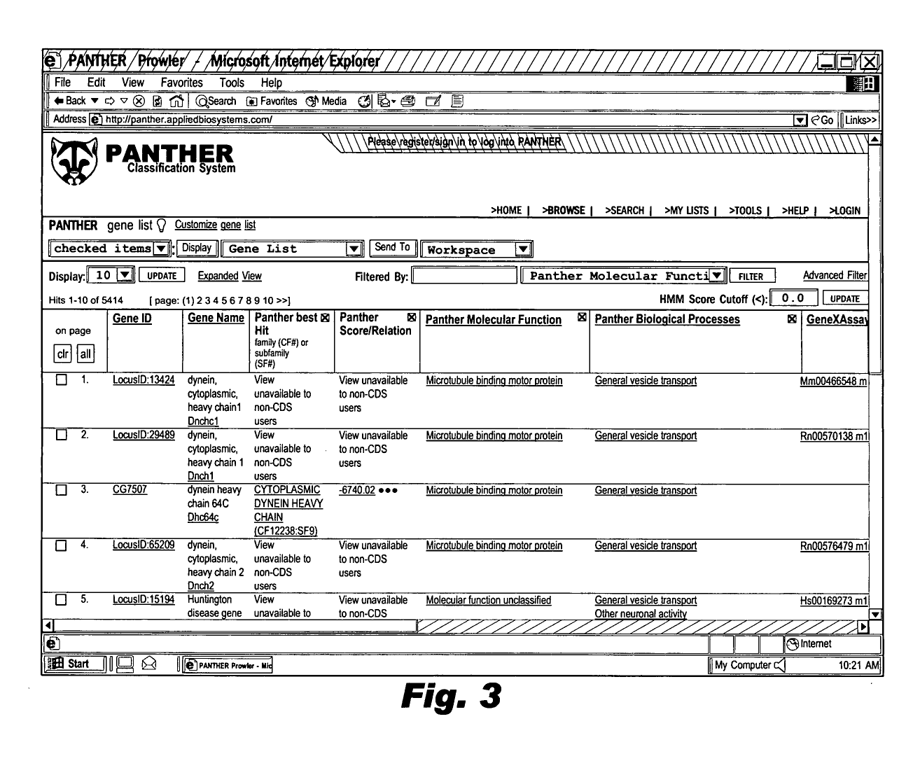

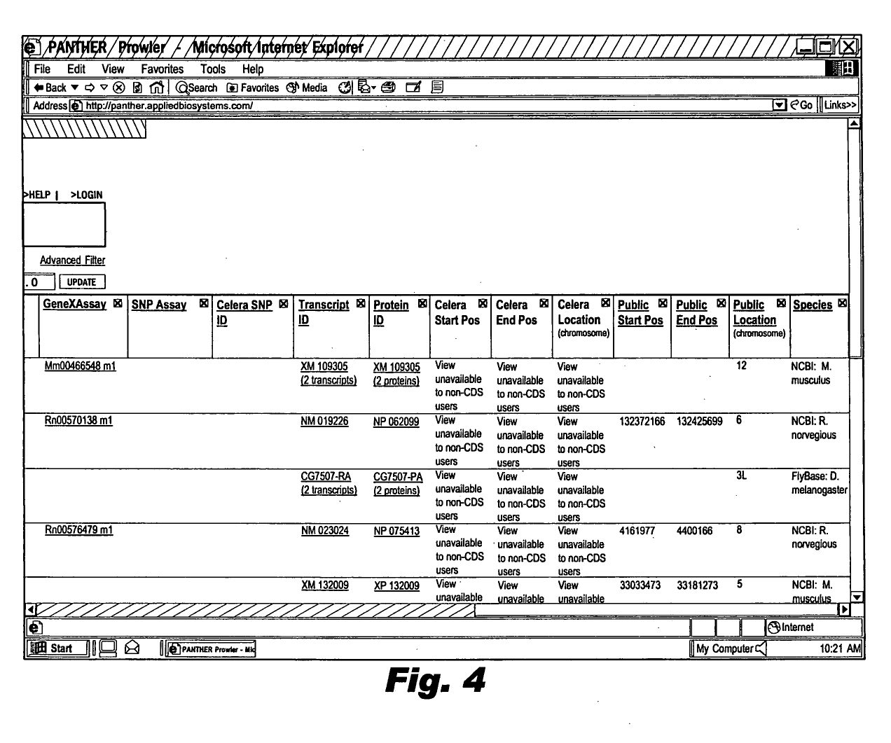

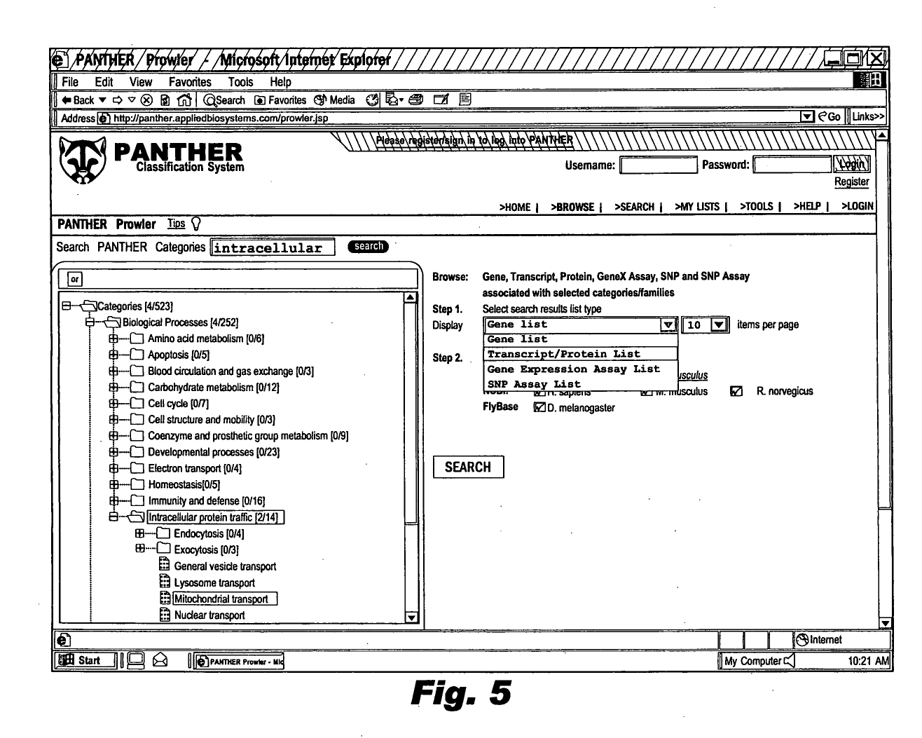

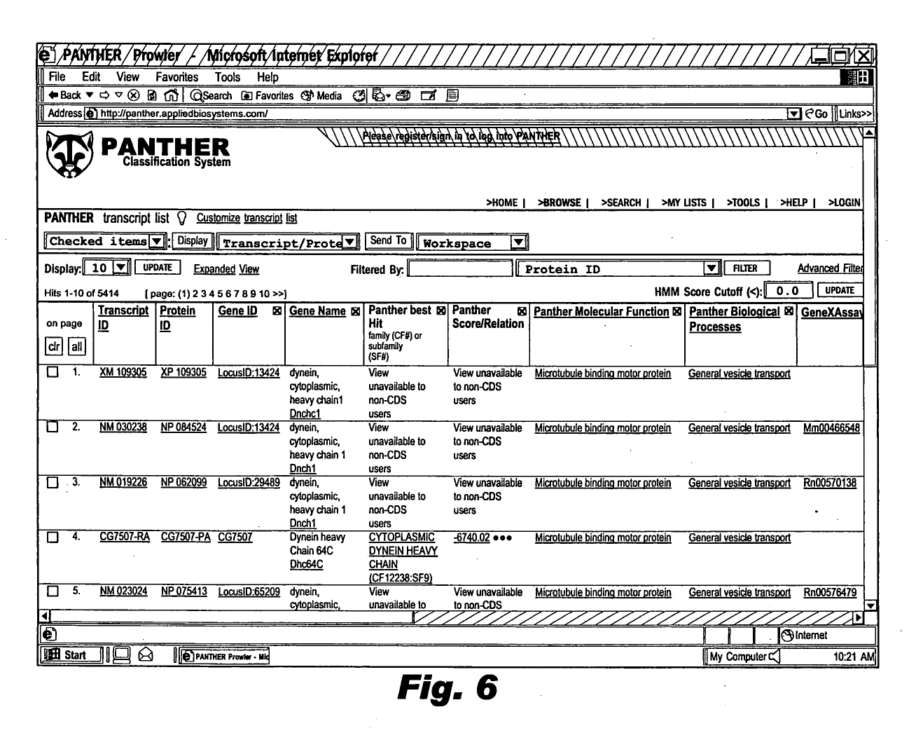

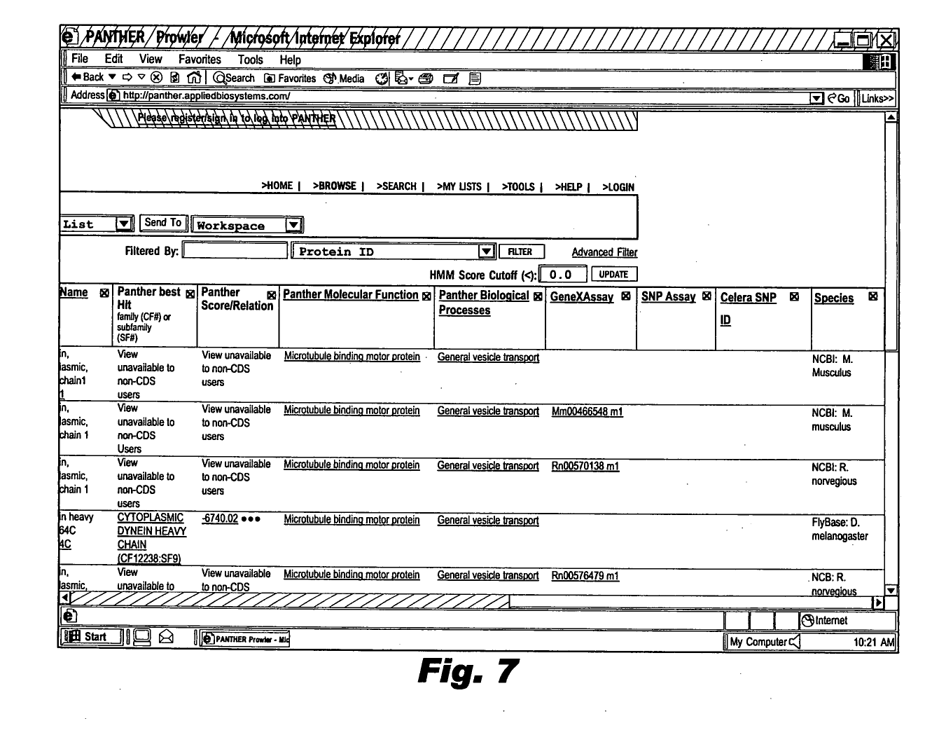

Once families, subfamilies, categories, and subcategories are selected, users can specify a Boolean operator 76 and a set 78 of sequence databases. Accordingly, recognizer 74 scores models 56 related to the selected families, subfamilies, categories, and subcategories against contents of the selected set 78 of databases comprising a subset of data 58. Matching multiple sequence alignments that fulfill the conditions of the boolean operator 76 are retrieved and communicated to the users as at 80. Since gene and protein sequences correlate, users may select to view a gene list as illustrated in FIG. 2, or a transcript/protein list as illustrated in FIG. 5. The gene list view illustrated in FIGS. 3-4 provides hyperlinks to gene data, while the transcript/protein list view illustrated in FIGS. 6-7 provides hyperlinks to protein data.

It is envisioned that various embodiments of the present invention may be implemented. For example, it is possible that pointers may be permanently instantiated between multiple sequences and the categories, subcategories, families, and/or subfamilies. Labeling sequence locations with functional and/or familial descriptions and sequence sizes is one way of accomplishing these pointers. In such cases, the statistical models may be discarded or only used periodically to update new sequence entries. Recognizer 74, may therefore equivalently use these pointers on a routine basis, and/or Blast input sequences to find appropriate categories, subcategories, families, and/or subfamilies. Some of these embodiments are explained below in greater detail. It should be readily understood that characteristics, components, and uses of these embodiments so described may be combined in various ways that will become readily apparent to those skilled in the art based on the preceding and subsequent disclosure.

In some embodiments, the browsable database can be a system for classifying, and predicting the functions of protein sequences in the context of phylogeny. Accordingly, the database may define a controlled vocabulary for protein annotation, as well as a method for classifying new sequences.

By way of overview of these embodiments, the browsable database library may contains over 2,200 alignments of related protein sequences (protein families), potentially containing a total of 188,000 non-redundant sequences from a variety of organisms. These families may further subdivided into nearly 40,000 subfamilies of closely related protein sequences.

Curators can be employed to accomplish the aforementioned organization. For example, every family and subfamily can be reviewed by a team of expert biologist curators. Also, every family and subfamily may be labeled by curators according to the most accurate name that applies to all sequences in the group. Every family and subfamily may be classified by (1) the molecular function(s) shared by the sequences in the group and (2) the biological process(es) in which these proteins participate.

Every family and subfamily may be represented as a statistical model (Hidden Markov Model or HMM) that describes the shared characteristics (“signature”) of the sequences in that family or subfamily. The database HMMs can be used to score all protein sequences predicted in a given genome (such as human and mouse), and therefore give a probabilistic prediction of the protein's (1) name, (2) molecular function(s), and (3) biological role(s).

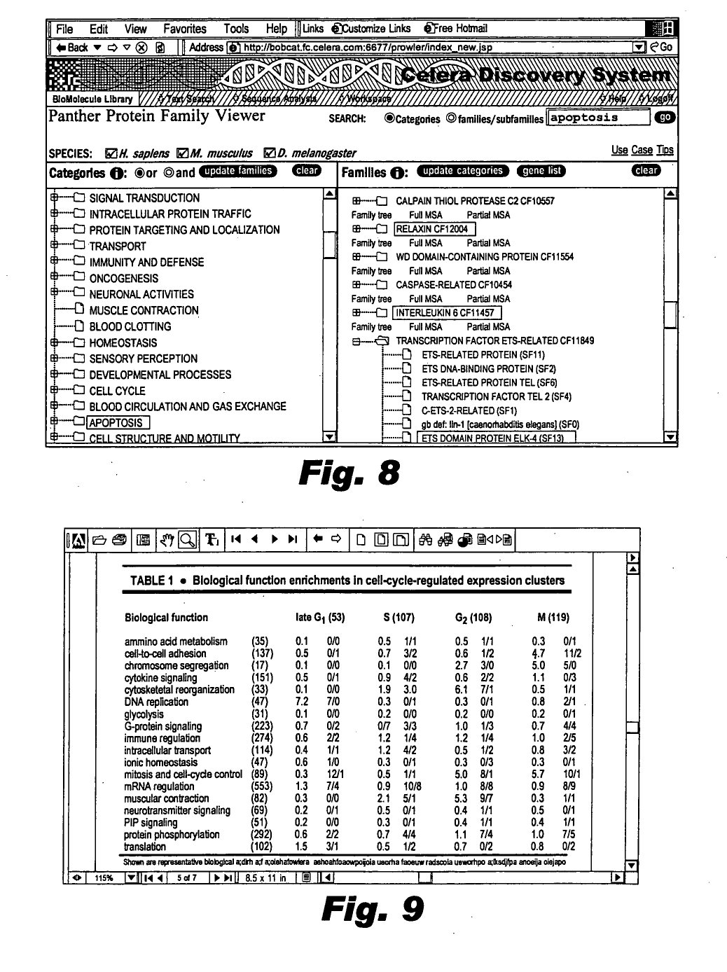

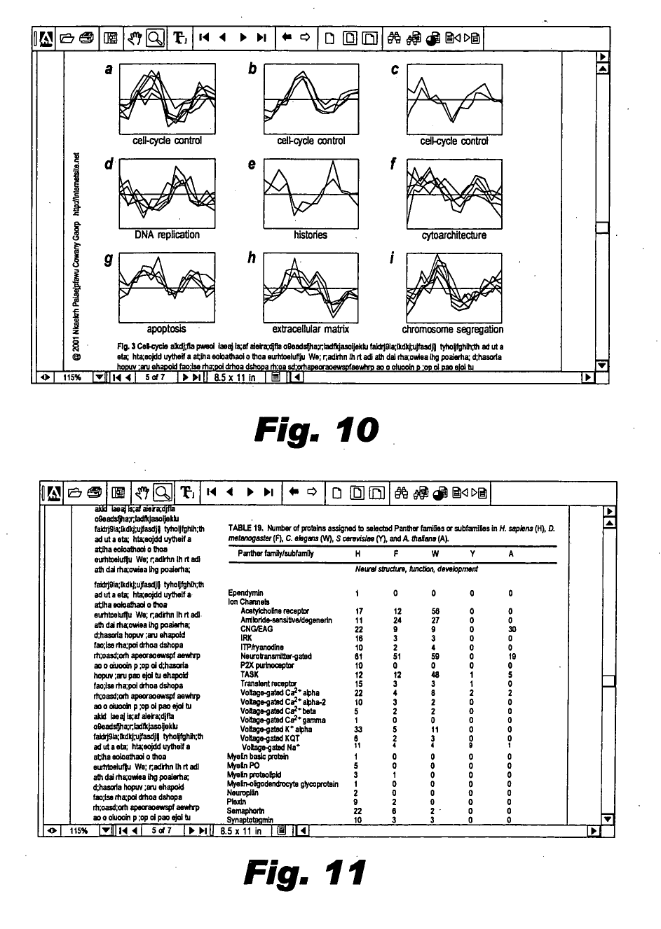

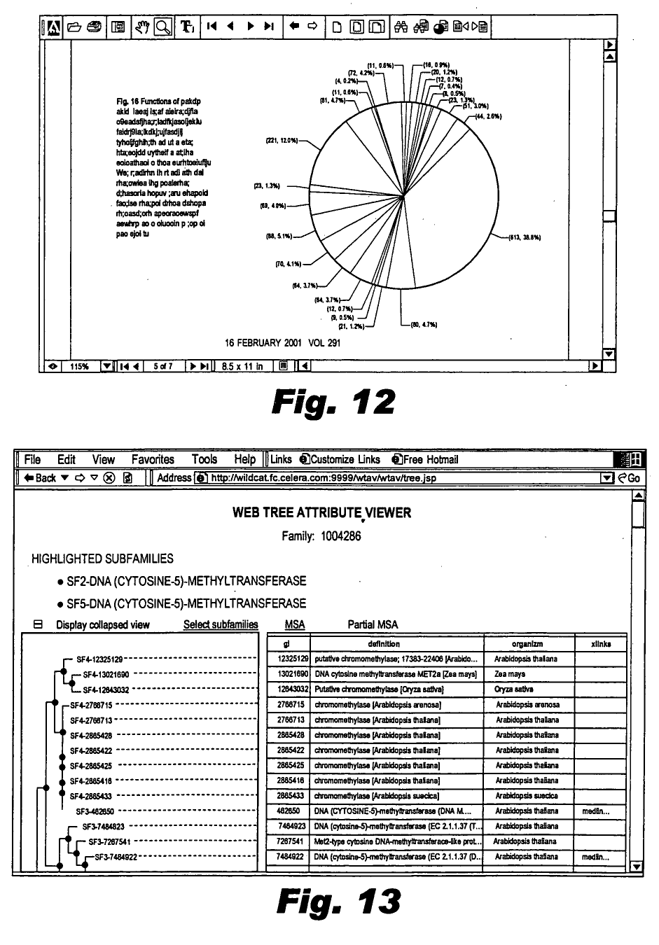

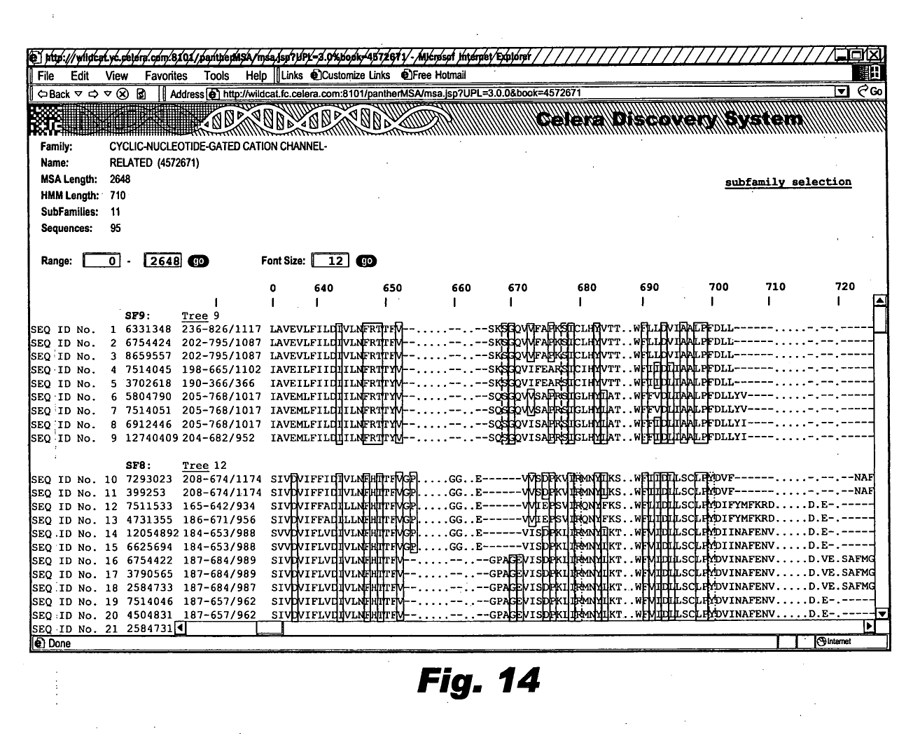

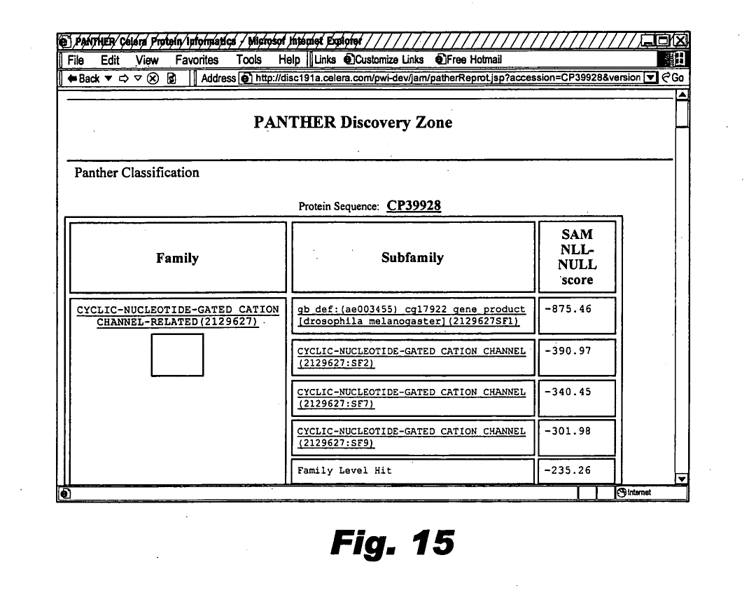

These embodiments have a variety of uses. One such use may be browsing the proteins predicted by in the human and/or mouse genomes as illustrated in FIG. 8. For example, a user might want to quickly locate all ligand-gated ion channels. Proteins can be browsed either by molecular function or by biological process. Another such use may be creating lists of proteins based on: (1) evolutionary relationships at the family level (e.g. all trypsin-like serine proteases) or subfamily level (e.g. chymotrypsin); (2) molecular function(s), e.g. all proteins predicted to be proteases; and (3) biological process(es), e.g. all proteins predicted to be involved in neuronal development. A further such use may be aiding analysis of mRNA and/or protein expression results as illustrated in FIGS. 9 and 10, which demonstrate examples from Cho et al., Nature Genetics 2001 and Cho & Campbell, TIGs 2000. For example, expression-based clusters can be correlated with biological processes. Also, gene products of certain target classes can be identified. A still further such use may be facilitating comparative genomics analysis. Predicted proteins from different organisms can be compared by family/subfamily relationships (orthology and paralogy) and by functions and processes. This kind of analysis can be found in the Human Genome paper (Venter et al., Science 2001). As another example, missing genes in a biosynthetic category for a microbe may suggest auxotrophic requirements. A yet further such use may be exploring protein family/subfamily relationships in the library of phylogenetic trees. These views as illustrated in FIG. 13 include both Celera-assigned subfamily annotations and Swissprot- and Genbank-assigned sequence-level annotation. Another such use may be exploring amino acid-level determinants of function and specificity as illustrated in FIG. 14. The library of multiple sequence alignments may highlight positions that can be conserved across an entire family as well as subfamily-specific positions. FIG. 15 illustrates a still further use, enhancing BLAST results. The database classification may be applied to organize by family/subfamily any protein-based BLAST search. This application can drastically reduce the amount of data to sift through (only one sequence per subfamily may be shown since they all have the same function) as well as provide additional information from the database classification.

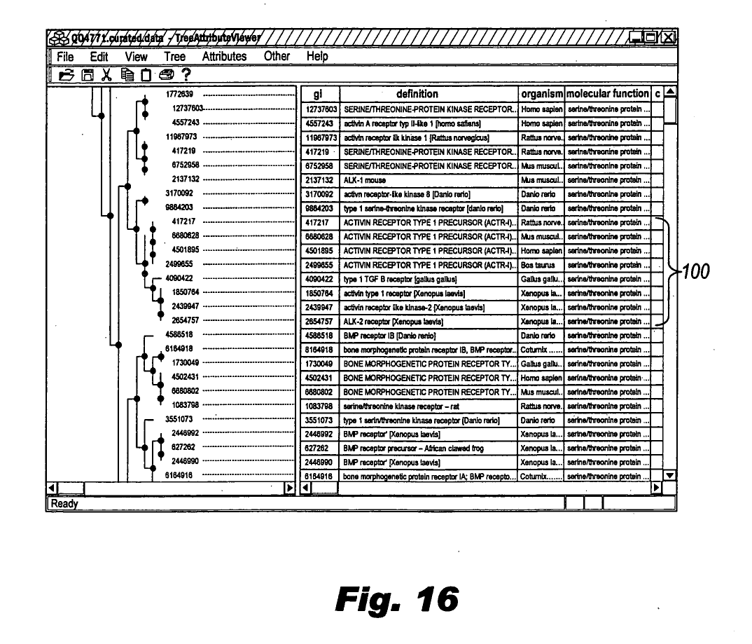

The browsable database terminology may be quite specific. For example, a family may be defined as a group of sequences for which a “high quality” (defined below) multiple sequence alignment can be generated. This may be helpful for building a phylogenetic tree, as well as for analyzing the multiple alignment for conserved and variant positions as a function of phylogeny. In this respect, database families can often be “tighter” (i.e. composed of more closely related sequences) than a Pfam family. Among the most extreme examples of this may be the representation of rhodopsin-class G protein-coupled receptors (GPCRs) in the database versus Pfam. In Pfam, this broad class of receptors may be represented by a single statistical model, 7tm_{—}1. The alignment that results from this model, however, may not contain enough information to accurately reproduce the phylogenetic and functional relationships between these receptors. In the database, this class may appear as twenty separate “families”. One may use a number of numerical measures, including subfamily-representative pairwise identity, and the number of conserved columns in an alignment, to automatically assess alignment quality. One may also use expert assessment of the resulting phylogenetic trees. If an alignment fails any of these measures, the family may be made still more restrictive as illustrated in FIG. 16.

Another key concept may be the idea of “subfamily.” A subfamily may be defined as a subtree of the family tree, all of whose sequences share an “attribute” in common. In the browsable database, one can use an arbitrary number of attributes to divide the tree into subfamilies. In the current library, the attributes used to define subfamilies can be nomenclature (often related to molecular function), molecular function category, and biological process category. For example, in the biogenic amine family, histamine H2 receptors can be a distinct subfamily from serotonin HT1A. In this case, the subfamilies can be defined by substrate specificity. In the HSP20 family, there can be different subfamilies for alpha-crystallin (molecular function: eye structural protein) vs. HSP27 (molecular function: chaperone).

The equation of subfamily with subtree may be helpful. One potential goal may be to define subgroups that share a pattern of amino acid conservation that differs from any other subgroup in the tree. This allows identification of a specific “signature” that can distinguish group from each other. Furthermore, the amino acid positions that define this specificity can be likely to be among the molecular determinants of that specificity. Because the tree can be built using a distance metric related to HMM-profile scoring (the same type of scoring used to score new sequences against the library), subtrees can be virtually guaranteed to have an amino acid conservation profile that may be distinguishable from that of any other subtree. In this way, the conservation profiles of different subfamilies can be compared to suggest the residues that may play a part in differing specific functions. These profiles can also be used to predict the subfamily of novel sequences (see HMM scoring below).

The equation of subfamily with subtree may be also helpful for inferring functions of related proteins. Again, how similar two proteins must be in order to infer the function of one from the other depends on the family and the function. A phylogenetic tree provides a framework for making that inference. Generally speaking, one has much more confidence when inferring the function of a protein that may be surrounded on both sides in a tree by proteins that share a function in common. In other words, one can make inferences based on consistency of annotation across a subtree and not on a single annotation.

The tree illustrated in FIG. 16 shows the activin receptor type 1 subfamily at 100. In some embodiments, subfamilies can be displayed in different colors. As seen in the “Definition” column, orthologs of this gene have been named in different ways in Genbank (lower case) and Swissprot (upper case) but should all obviously share the same nomenclature.

Another browsable database term may be “category.” This term refers not to a sequence-derived property such as family or subfamily, but to a category in a classification schema such as GO (Ashburner et al., Nature Genetics 2000). In the database, categories can be labeled according to the type of classification (molecular function or biological process) as well as the “level” or depth of the category. For example, receptor may be a level 1 molecular function, and G protein-coupled receptor may be a subset of receptor and a level 2 molecular function. The more detailed the classification, the deeper the level. Because biology may be not simple, a given family or subfamily may be assigned to more than one category, or a particular category may have more than one parent category. In the database schema, a given category will always appear at the same level, in order to facilitate navigation. For example, nuclear hormone receptor may be a level 2 category that may be both a child of receptor (level 1) and transcription factor (also level 1). It may be not a child of another level 2 category, or a level 3 category for example, as seen in the current release of GO. That said, the database schema adheres very tightly to the GO schema, although database schema diverges in some areas from GO. Except for these areas, the database schema may be essentially a subset of GO (most categories that can be omitted can be very detailed or redundant categories in GO). The goal of the database schema may be to allow a user to rapidly browse a large sequence database, and to create lists of genes based on functions or families of interest. In the more detailed categories of GO, very few proteins appear in a given category, so these categories generally do not create efficient gene lists and complicate navigation with too many possible paths.

In the database, families and subfamilies can be linked to categories via expert curation. The overall process for building the database classification may be includes several steps. The basic steps are: (1) family clustering; (2) MSA, family HMM and phylogenetic tree building; (3) family/subfamily definition; (4) subfamily HMM building; (5) molecular function assignment; and (6) biological process assignment. Of these steps, (1), (2) and (4) can be computational, and (3), (5) and (6) can be human-curated (with extensive aid of software tools).

The first step in the database library-building process may be to cluster protein space into families, and several sub-steps can be included. For example, seed selection involves choosing the proteins that will serve as “seeds” around which initial HMMs can be built. The database of all known proteins may first be split into clusters defined by a percent identity (25%) and length based (70-130%) cutoff. This sub-step allows each cluster to contain related proteins that can be all of roughly equal length, so that they can be likely to share the same domain structure. In some embodiments, the clustering may be begun with Genbank NR Protein Release 122 (Feb. 15, 2001), after first removing sequences annotated as partials or mutants. From each cluster, a representative seed was defined as the sequence closest to modal length for the cluster. This definition may be also helpful given the heterogeneous quality of public sequence databases, since it assumes that the most common length may be most likely to be “correct”—i.e. it may be neither a fragment nor a potential chimera.

Another sub-step may be initial cluster building. The goal of this sub-step may be to generate a cluster of sequences that can be globally homologous to the seed, in order to generate the initial HMM to reflect the seed's domain arrangement. In this sub-step, the seed may be BLASTed against the “filtered” NR database to bring in additional relatives. It may be helpful to first “filter” from NR any known sequence fragments, sequences that can be exact subsequences of other NR sequences (these too can be likely to be fragments) and sequences annotated as mutant, engineered or chimeric proteins (these will weaken the residue conservation profiles since site-directed mutants can be generally in functionally relevant positions). In this sub-step, an E-value cutoff (10\hat{ }−5) may be used rather than a percent identity score, the same length cutoff may also be enforced as in seed selection. All related sequences passing these thresholds may be brought into the initial cluster.

A further sub-step may be extended cluster building. The goal of this sub-step may be to extend the clusters to include as many related sequences as possible. This sub-step (1) makes the resulting HMMs much more powerful since there can be more “observed” sequences to derive residue substitution statistics, and (2) brings more sequences into the phylogenetic trees, providing as much information as possible about relationships that biologist curators can use to infer function.

The initial cluster may be used as input into the buildmodel procedure of the UCSC SAM 2.0 package (Karplus et al., 1998). Sequences can be weighted relatively using the Henikoff weighting scheme (Henikoff & Henikoff, 1991), and given an absolute weight using the formula nseq(1−<Pmax>), where nseq may be the number of sequences in an alignment and <Pmax> may be the average probability for the most common amino acid at each position. This weighting scheme was tested extensively by UCSC in the CASP2 competition. Because it would be computationally prohibitive to score the resulting HMM against the entire NR protein set, one may need to define a smaller “search set” of proteins that can be potentially related to the seed. The seed may be used to run PSI-BLAST for three iterations, and the search set may be defined as the set of all proteins that appear in any of the PSI-BLAST iterations (not just the final iteration, since PSI-BLAST can “wander” to very different protein families). The initial HMM may then be scored against the search set. There may be no length restriction to hits here—any protein may be brought into the cluster if it shares even a local (partial) match to the HMM as long as the resulting alignment may be of high quality. Empirically, one may find that for most families a related protein that has an NLL-NULL score better than −100 (units can be natural logarithms or “nats”) has a high-quality alignment, and sequences scoring better than this cutoff may be added to the initial cluster to define the extended cluster. One may also find, at this stage, that it can be beneficial to search for new cluster members using not only the family HMM, but subfamily HMMs as well. The HMMTree algorithm, when it builds a tree for a family, also cuts the tree into subtrees (i.e. subfamilies) based on information theory (see below for details). Intuitively, this can be thought of as defining all of the subgroups in the family which, if left separately, contain information that may be lost if they can be combined into a single statistical model. Therefore the subfamily HMMs sometimes recognize related family members that the family HMM does not.

The goal of the MSA building and HMM reestimation stage may be to obtain a multiple sequence alignment for the extended cluster, and to reestimate the parameters of the HMM given all of the new sequences brought into the cluster during the extension step. Accordingly, the initial model and extended clusters can be used as input to the SAM modelfromalign procedure. Sequences can be aligned (using SAM aligntomodel) to the highest scoring HMM from the initial cluster (either the family HMM or a subfamily HMM) to produce a multiple sequence alignment. Recall that the extension process can bring in proteins that only match locally (over a single region, such as a domain) if the match may be close enough to pass the score threshold. Therefore it may be helpful that this alignment step be a local-local, or Smith-Waterman, type of alignment. Sequences can be then re-weighted as above, and these weights can be combined with the alignment to produce a reestimated family HMM. However, unlike in extended cluster building, in the modelfromalign procedure here, the model topology (i.e. number of match states) may be fixed—it may be constrained to remain the same as in the initial model. If the model topology were allowed to change, the motif or domain that most of the sequences have in common would determine the model topology, which can result in poor statistical models for the cluster.

It may be helpful that the MSA be of high quality. Garbage in, garbage out: if the alignment may be of low quality, it may be difficult to build an accurate tree, and therefore nearly impossible to infer the relationship between function and phylogeny for a given family. Therefore, it may be at this step in the process that one may choose to assess the quality of the MSA. A number of automatic criteria may be defined for flagging potentially poor alignments. If an MSA does not pass these thresholds, the family-building process may be restarted around the seed using a more stringent BLAST E-value cutoff (10\hat{ }−20). One may find that about 5% of the UPL3 families fail this first QA step and must be rebuilt. If the cluster still fails the QA check after being built a second time, it may be sent to the queue for building by hand.

The phylogenetic tree building method uses HMM scoring to define a distance between clusters during an agglomerative clustering process. For each cluster at any step in the process, a statistical profile may be built that describes those sequences. In this way the algorithm builds up a statistical description of relevant positions in the cluster, and preferentially joins the group to other groups that share the same conserved positions. The distance between any two clusters may be defined as the average HMM score of the sequences in A versus the profile for B, added to the average HMM score of the sequences in B versus the profile for A. The two clusters that have the maximum value of this function can be joined. If the sequences in group A all score well against the profile for B, and vice-versa, then the groups have similar residue conservation patterns and should be joined. Branch lengths for the join can be estimated using symmetrized total relative entropy (see Sjolander, ISMB Proceedings, 1998).

A key feature of the new HMMTree algorithm may be how it handles local matches, since not all members of an extended cluster will necessarily align globally. This may be helpful since sequence fragments and chimeric sequences, as well as domain-level matches, can be common in current databases. Therefore, the distance function may be scaled according to the length of the match between a sequence and a profile without penalizing partial (local) alignments.

Automatic prediction of subfamilies may be accomplished using sequence information to attempt to predict automatically how protein families should be divided into subfamilies. The goal may be to give the curators both a headstart (to make their jobs easier and less tedious) and to provide a guideline that may be roughly consistent across different families. To do this, one may take advantage of the observation that if two groups share the same conserved residues they often have the same functions; conversely, different conservation profiles correlate with different functions. Intuitively, a related algorithm may find the nodes in the tree where two subtrees having “significantly” different profiles can be joined together and suggest that each of these subtrees should be a separate subfamily. Such an algorithm may be described in detail in (Sjolander, doctoral dissertation 1997).

The family clustering procedure described above naturally produces overlapping clusters for many protein superfamilies. One potential goal for clustering was to span protein space well, not necessarily to partition it such that each sequence can appear in only one family. Because of the domain arrangement of proteins, as well as the broad evolutionary distances spanned by some families, the rigorous partitioning approach does not provide as much context as the spanning approach. However, one may want to remove any clusters that can be essentially completely contained in other clusters. Thus, the method may include removing overlapping clusters by sorting the clusters from largest to smallest, and then going down this list asking if >90% of the sequences in the nth cluster can be contained in the set spanned by the (n-1) accepted clusters. If so, then the nth cluster may be removed from the set. Because of this criterion, there can be a number of examples of overlapping database families.

After the phylogenetic trees can be built, they can be reviewed and annotated by a team of expert curators. Unlike previous approaches toward curation, the present approach to curation may be performed in the context of a phylogenetic tree; i.e. a family of sequences can be annotated in the context of the set of (nearly) all related proteins. This allows curators to make inferences that could not be made if they were looking at a single sequence at a time, as well as perform consistency checks on the incoming data as well as the annotations they make themselves. Also, unlike the approach adopted by Proteome, Inc., most families can be reviewed by curators who have expert knowledge of the relevant family, molecular function or biological process. This may result in additional inferences that can be more likely to be accurate.

One of the curator's tasks may be to review the position of the automatic subfamily assignments. In other words, his/her task may be to ensure that the tree may be divided into subtrees such that each subtree contains sequences that share: (1) the same name (or a consistent name can be applied to all sequences in the subtree); (2) the same molecular function; and (3) the same biological processes. If an automatically chosen subtree meets the above criteria, it does not need to be changed. (A curator may choose, if several neighboring subfamilies can be annotated consistently, to move a subfamily node upstream, toward the root of the tree). If a subtree does not meet the above criteria, it must be broken into consistent subtrees. Note that not all sequences must be individually annotated in exactly the same way for the curator to decide that they all, in fact, can be likely to share the same attributes. In fact, the lack of standards for nomenclature, the wide range of annotation quality and the years of transitive sequence annotation have made biologist interpretation an imperative. The curator's ability to infer the functions of proteins that can be either incorrectly or inadequately annotated may be advantageously exploited. Putting the sequences into a phylogenetic context may be a powerful means of grouping sequences together. If an unannotated sequence may be surrounded on all sides by sequences known to have a particular function, it may be very likely that this unannotated sequence shares that function as well.

The annotation process has a carefully defined protocol, and set of software tools to facilitate it. One tool may be the database “tree-attribute viewer.” This tool displays a protein family phylogenetic tree together with a table containing sequence-level annotations for each sequence in the family (mostly derived from SwissProt and GenBank). Each of the fields of the table has one or more links to more detailed external information, including PubMed abstracts. There may be also an internal Tracking Database that contains information about the curation process for each family, including the name of the annotator, the date of annotation, any problems or outstanding issues uncovered during curation, etc.

The curators of the database families can be selected based on areas of expertise. In addition to the in-house biologists at Celera, 23 different biologists (mostly from Stanford and UC San Francisco) have been brought in to annotate the families. In addition to reviewing the membership of sequences within a subfamily, the expert biologists gives each subfamily a biologically meaningful name. In some cases, all sequences within a subfamily have the same definition, so naming the subfamily may be trivial. Often, different synonyms may have been used for each of the sequences in a subfamily. In that case, the curator will use their expert knowledge to pick the most informative name. If a SwissProt sequence may be present in a subfamily, that name may be often chosen because of its high quality. An effort may be made to maintain a naming convention across subfamilies within the same tree and between different trees.

Often there can be subfamilies where none of the individual sequences has a clear function. However, that subfamily may be present in a family because there may be significant sequence similarity with other subfamilies. The phylogenetic tree and Multiple Sequence Alignment give the expert biologist more information about the function of genes within a subfamily that was likely available to the people who originally named the sequences. The convention used for naming these subfamilies may be to determine the closest subfamily whose function may be clear (X), and to name the uncertain subfamily “X-RELATED.”

Information about the organisms from which the sequences derive may be also useful in naming subfamilies. It may be not uncommon for a tree to contain orthologs from a wide variety of organisms. In this case the naming may be often inconsistent (often due to organism-specific naming conventions), but it may be clear from the MSA and tree that all sequences can be orthologs. In this case a name may be picked that may be most biologically informative, and all subfamilies can be given the same name. This rule may be not applied universally because sometimes there can be well known names in different species that the curator may be uncomfortable overwriting.

Biologically meaningful names can be also given to each of the families. Occasionally, a family will have subfamilies that all have the same name. In this case, the family name may be the same as the subfamily names. Usually, there can be several different functions across subfamilies of an evolutionarily conserved protein family. If the protein family has a well established name, then the database family may be given that name (eg. ANTP/PBX FAMILY OF HOMEOBOX PROTEINS). Often there may be no well-established name. In this case, the curator either gives the protein a more general name that applies to all proteins in a family (e.g. NUCLEAR HORMONE RECEPTOR) or finds the most common subfamily name (Y) and names the family “Y-RELATED.”

The method further includes making a schema for molecular function and biological process classifications. One of the largest benefits of classification may be that genes can be placed into a defined schema having a controlled vocabulary. This classification allows one to query genes in an efficient manner.

The publicly supported Gene Ontology (GO) has been available for a few years, and continues to mature. The GO schema captures complex relationships between genes and their biological functions, and has many different categories. In many cases it can be difficult to navigate the GO system because of its sheer size. GO was designed primarily to provide a consistent nomenclature for the annotation of gene products.

A more streamlined version of GO may be desirable for several reasons. First, it may be helpful a classification that may be easier to navigate. For example, in the GO biological process schema there can be a total of 3994 unique categories. These can be arranged into a directed, acyclic graph (meaning a child can have more than one parent), and if the number of categories may be counted once for each subtree it appears in, there can be 7568 categories to navigate. Furthermore, these categories can be arranged to be up to 12 levels deep, again making navigation difficult. For comparison, the database molecular function schema may contain contain two-hundred forty-nine unique categories and be three levels deep. It may be helpful to have a schema that may be not too deep, and in which depth iss indicative of annotation specificity. Second, the database was designed to help rapidly make lists of genes using three different criteria: (1) family (or subfamily), (2) molecular function category, or (3) biological process. The goal may be to get to a level that may be specific enough to retrieve a list small enough to sort through, but not so specific that only one or two gene products appear there. The database schema has adopted many of the higher-level GO categories to make the classifications as compatible as possible, and to allow one to “toggle” between viewing the database and GO. Another point may be that database contains categories not found in the latest version of GO. Many of these categories were introduced because some families containing mammalian proteins could not be classified into any GO categories. One example may be the datbase's viral protein category for classifying endogenous viral proteins. In cases where GO may be missing categories, the database team consulted with experts to expand the classification system. The database team may be working with the GO consortium to ensure the compatibility of the two schemas as they evolve.

The database schema may be composed of two types of classifications: molecular function and biological process. The molecular function schema classifies a protein based on its biochemical properties, such as receptor, cell adhesion molecule, or kinase. The biological process schema, on the other hand, classifies a protein based on the cellular role or process in which it may be involved, for example, carbohydrate metabolism (cellular role), signal transduction (cellular role), neuronal activities (process), or developmental processes (process). Oncogenesis is, in fact, a pathological process, but since it may be field receiving much attention, it may be included in the database biological process schema.

In some embodiments, there can be no more than three levels of categories in either database schema. Level 1 categories can be broad and general functional terms, such as receptor, protease, or transcription factor in the molecular function schema, and carbohydrate metabolism, signal transduction, or developmental processes in the biological process schema. Level 2 and 3 categories can be subcategories of level 1 categories, and can be more specific functional terms, such as G-protein coupled receptor, serine-type protease or zinc finger transcription factor in the molecular function schema, and glycolysis, MAPKKK cascade or neurogenesis in the biological process schema. Under parent categories having more than one child, an “other” category may be introduced, such as other receptor or other carbohydrate metabolism process, to avoid generating an excessive number of categories with few subfamilies classified in them.

One point may be that, properly speaking, the ontology may be a DAG (directed acyclic graph) rather than a true hierarchy. In practice, this means that a given category can have more than one parent. For simplicity, one may attempt to minimize the number of instances in which the schema deviates from a hierarchy, but there can be still many cases where a child category has multiple parents. Unlike the full GO schema, a child must appear at the same level under each parent so that depth corresponds to specificity. For example, nuclear hormone receptor (level 2) may be classified under the parents receptor (level 1) and transcription factor (level 1).

The method further includes assigning families and subfamilies to categories. After extensive work was done to create/adopt classification systems for both molecular functions and biological processes, expert curators were again brought in to classify subfamilies according to their function. Curators use many different pieces of information while performing the classification, such as textbooks, Medline abstracts, Swiss-Prot keywords and definitions, the database subfamily names, Entrez records, and their own expert knowledge of the field. Because they can be curating in the context of the phylogenetic tree, they may also infer function based on what may be known about adjacent subfamilies. Curators may only place subfamilies into one of the existing database categories; they may not create a new category unless it may be cooperatively decided that there may be a compelling reason to do so.

As most biologists know, enzymes having a common biochemical (molecular) function usually can be related proteins. The same may be often not the case for proteins participating in the same biological process—i.e. most pathways can be comprised of a series of different biochemical reactions. Likewise, molecular function changes much less dramatically within a phylogenetic context than does the biological process. Therefore, inferences about molecular function can more often be made than can inference about biological process. Again, knowledge of the biological context may be helopful. For example, an expert may be hesitant to infer the biological process of a serine/threonine kinase, but not that of citrate synthase. The number of pathways a biochemical reaction may be used in affects one's ability to infer biological process.

The method also includes assigning families to categories. After the subfamily-level classification was completed, categories were assigned to the family level models. Since many families contain subfamilies with diverse functions, only the categories that were common to all subfamilies were assigned to the families. This is, of course, more pronounced for biological process categorization than molecular function. It may be therefore possible for a family to have no assignable category at all, even if there can be a number of assignable subfamilies. This means that any sequences that can be recognized by the HMMs as belonging to a family but not a specific subfamily (i.e. this may be a novel subfamily not represented in the database library), will not be classified to the ontology even though they can be associated with a family. This may be a very helpful point, because it prevents the database from making the kind of transitive errors of assignment that can plague other methods.

After the initial classification effort, all the assignments underwent a rigorous QA process, which may be divided into two separate steps:
(1) validation and (2) consistency check. During the validation step, experts may review all subfamily assignments in each category. That is, rather than making classifications family by family as in the initial assignment process, classifications were checked category by category, generally by experts with knowledge of the relevant area. In cases that were not obviously correct, textbooks, Medline, and other available tools were used to resolve discrepancies. If a subfamily was incorrectly classified, or was not classified in a category in which it belonged, reviewers were encouraged to provide reclassifications. These classifications may be reviewed and subjected to QA also. After the validation step was completed, a consistency check was performed. Subfamilies that shared common sequences but had not been consistently classified across different families were reviewed. Depending on the context of the subfamilies, the reviewer would decide whether to make them consistent. For example, if 4 sequences were shared by two subfamilies with 5 sequences each, these two subfamilies should have basically the same classification. However, if 4 sequences were shared by two subfamilies with 5 and 200 sequences, the functional classification of these two subfamilies could be different (one might be much more specific than the other). Only subfamily assignments that pass the QA process appear in the Discovery System.

The method continues with classifying a set of sequences. Although a version of the database library may have been built using only publicly available sequences, the statistical models in the library can be used to accurately classify novel protein sequences as well. In other words, the database provides not only a controlled vocabulary for protein annotation, but also a means for consistently applying the vocabulary to new proteins.

Every sequence in the “query” set may be scored against the database library of HMMs. The search takes advantage of the hierarchical structure of the library. Instead of scoring every sequence against all ˜42,000 family and subfamily HMMs, a sequence may be first scored only against the 2,236 family HMMs. If the family HMM score may be marginal or significant (such as an NLL-NULL score cutoff of −20), the sequence may be scored against the subfamily HMMs for that family. All HMM scores (family or subfamily) better than −20 can be stored in a database and can be retrieved in the browsable databse interface. For the purposes of classification, however, the highest scoring HMM (either family or subfamily) may be used. This may be one advantage of a browsable database, that a protein can be recognized as being a close relative of training sequences, or a more distant one, and that these two cases can mean very different things for the purposes of function prediction. In general, if the top-scoring HMM may be a subfamily HMM, then the query sequence belongs to that subfamily. This may be true because the subfamily HMM may be in competition with the family HMM that has many more examples to generalize from and will therefore score more highly for sequences that belong to a new subfamily (i.e. one not represented in the family alignment). This may be helpful because, for example, a novel serine/threonine kinase receptor family member can only be inferred to have only that general function, while a member of the BMPR1 subfamily can be inferred to be involved in the specific biological process of skeletal development.

The method further includes providing confidence levels associated with functional predictions. Lists of proteins predicted to be in a given family, subfamily or function class can be filtered using these confidence levels. For family and subfamily membership, confidence may be given quantitatively by HMM score. The “more negative” the NLL-NULL score, the more confident the prediction is. For most families, an NLL-NULL a score of −200 or less indicates a very close relationship with the training sequences and a very confident functional prediction. A score between −200 and −50 generally indicates a close relationship and a confident functional prediction. Scores between −50 and −35 can be usually still significant, but indicate a more distant relationship that often, but not always, allows accurate functional inference. Scores between −35 and −20 can be worth examining, especially when mining for novel members of an interesting family, but should be supported with additional analysis tools such as BLAST or Pfam. Some embodiments may have family-specific confidence cutoffs for the relatively few families to which these more general score guidelines do not apply. For some shorter proteins, such as cytokines, a score as poor as −20 may be nearly always significant, while for coiled-coil proteins such as myosin a score of −50 can still be misleading.

Some embodiments of the browsable database created as described above may be designed for high-throughput functional analysis of large sets of protein sequences (1). It may be used to annotate the human genome (2) as well as the Drosophila genome (3). Like databases such as Pfam (4) and SMART (5), the browsable database may use a library of Hidden Markov Models (HMMs) to annotate sequences with information from homologous sequences. However, unlike these databases, the goal of the browsable database may be not to annotate individual domains, but the overall biological function(s) of the molecule. Also unlike these other databases, because many protein families have branches that have diverged in function during evolution, the browsable database library may contain HMMs not only for families, but also for functionally distinct subfamilies. In these cases, subfamily annotation allows a much more precise definition of nomenclature and biological function.

The browsable database can be composed of two main components: a library and an index. The library may be a collection of “books”, each representing a protein family as a multiple sequence alignment, an HMM and a family tree. Functional divergence within the family may be represented by dividing the tree into subtrees (subfamilies) based on shared function, and by subtree HMMs. The index can be an abbreviated ontology for summarizing and navigating molecular (biochemical) functions and biological processes (such as cellular roles or even physiological functions). Families and subfamilies may be defined and named by biologist curators, who then may associate each group of sequences with terms in the index ontology.

Protein query sequences can then be scored against the functionally-labeled family and subfamily HMMs. Query sequences may be classified with the name and functional assignments of the best-scoring HMM, with the HMM score providing an estimate of the confidence level of the classification. Like other HMM-based approaches, the browsable database classification scales well for genome projects: the curated functional assignment may be performed up-front on sets of training sequences that span many organisms, and can then be transferred to other organisms using the labeled HMMs. As a result, the browsable database classifies a significantly larger fraction of human genes than does LocusLink (Table 1).

TABLE 1

| | LocusLink GO | Browsable Database |
|---|---|---|
| Molecular Function (NP) | 42 | 52 |
| Molecular Function (XP) | 0 | 19 |
| Biological Process (NP) | 41 | 46 |
| Biological Process (XP) | 0 | 17 |

Table 1 illustrates the percentage of human genes (approximated by LocusLink entries) having functional ontology classifications from the browsable database and from LocusLink GO associations. Percentages of genes classified can be shown for two sets of LocusLink entries: NP (with a curated RefSeq protein, accession beginning with NP, total: 13,780), and XP (with only a provisional RefSeq entry, accession beginning with XP, total: 38,506). The total number of LocusLink entries that hit an HMM of the browsable database may be 9276 (67%) for NP, and 9141 (24%) for XP.

Some versions of the browsable database use the GenBank non-redundant protein database to define sets of training sequences for HMMs. These HMMs can be used to classify human gene products from LocusLink, and Drosophila melanogaster gene products from FlyBase. Additional versions include training proteins from the sets curated at Celera, with additional HMM scoring of Celera-curated human and mouse gene products.

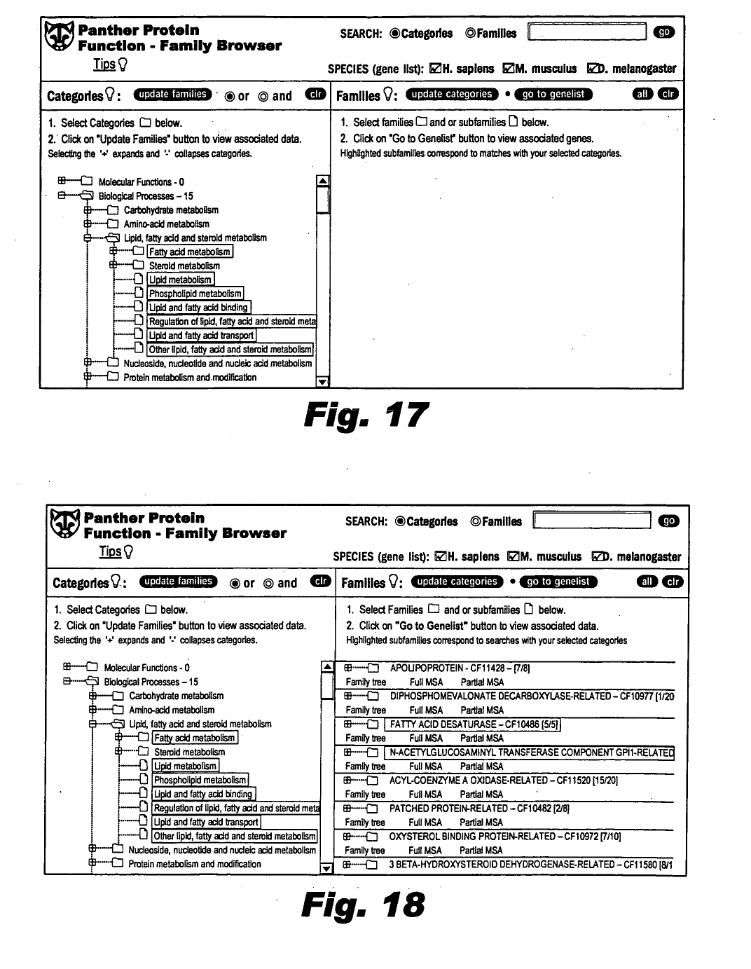

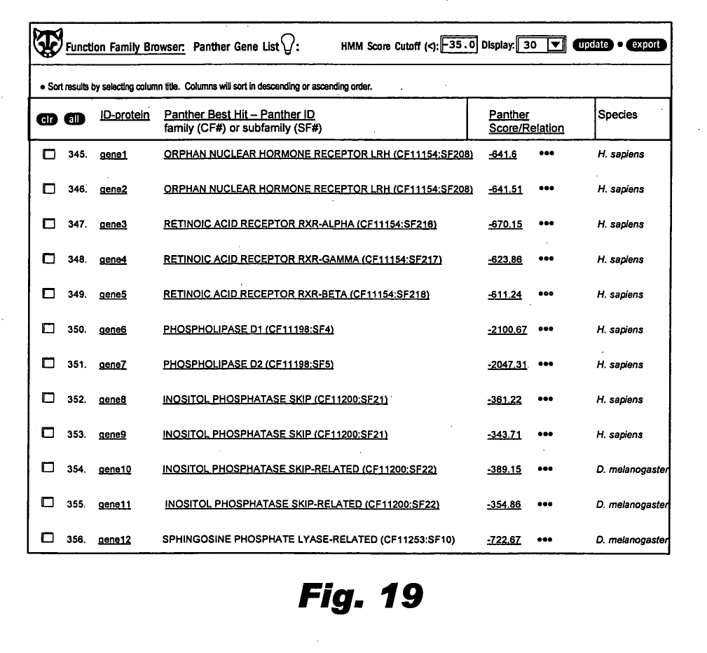

The browsable database may allow users to browse sequence database contents by protein functions, facilitating access to biologists. Browsing of controlled vocabulary terms can be much simpler than trying to construct effective queries in databases that have free text annotations. The primary entry point into the browsable database may be the browsable database interface, which may use a file-folder analogy to navigate index molecular functions and biological processes as illustrated in FIGS. 17, 18, and 19. An illustrated example of browsing the database by biological functions includes: (A) selection of biological process lipid and steroid metabolism in FIG. 17 (note that subcategories can be independently selected/deselected); (B) retrieval of protein families and subfamilies assigned by curators to the selected functional categories in FIG. 18; and (C) retrieval of a list of human genes encoding proteins that match the selected family and subfamily HMMs in FIG. 19. The index ontology may be essentially hierarchical (though, more accurately, it may be a directed acyclic graph as child categories occasionally appear under more than one parent if it may be biologically justified). The index may contain many of the same higher-level categories as the more comprehensive Gene Ontology (GO), and may be mapped to GO, but may further be arranged quite differently in order to facilitate navigation and large-scale analysis of protein sets. The index may also contain a number of vertebrate-specific categories that do not appear in the current release of GO, such as additional developmental and immune system categories.

After selection of a set of functions, the interface may retrieve the list of protein families and/or subfamilies that may have been previously assigned, by biologist curators, to those functions. A user can make further selections in the family/subfamily list, and then generate a list of proteins or genes that score significantly against the HMMs for the selected families and subfamilies. In some versions, gene lists may be available for LocusLink human genes, and FlyBase Drosophila genes. Gene lists can be sorted and easily exported in tab-delimited format.

In addition to browsing, the browsable database can be accessed by text searching of curator-assigned family and subfamily names, or of the GenBank identifiers or definition lines of training sequences. Training sequences for the classification can also be searched by BLASTP.

According to some embodiments, data may be available to support the curated classifications, including phylogenetic trees, multiple sequence alignments, and sequence annotation. The multiple sequence alignments used to generate the phylogenetic trees can be downloaded and viewed in an HTML viewer. One of the features of the MSA viewer may be that it highlights not only family-conserved columns (amino acids conserved across the entire family), but also subfamily-conserved columns (amino acids conserved within a subfamily but not found in other subfamilies). Curator-defined subfamilies may have distinct annotations and often distinct functions, so these subfamily-conserved columns may provide hypotheses about which residues may mediate functional divergence or specificity as illustrated in FIG. 20. Specifically, FIG. 20 illustrates the browsable database multiple sequence alignment view, highlighting globally conserved positions 102, and subfamily-specific conservation patterns 104 that may indicate residues helpful for functional specificity. Pfam domains may be shown as bars 106, one for each subfamily.

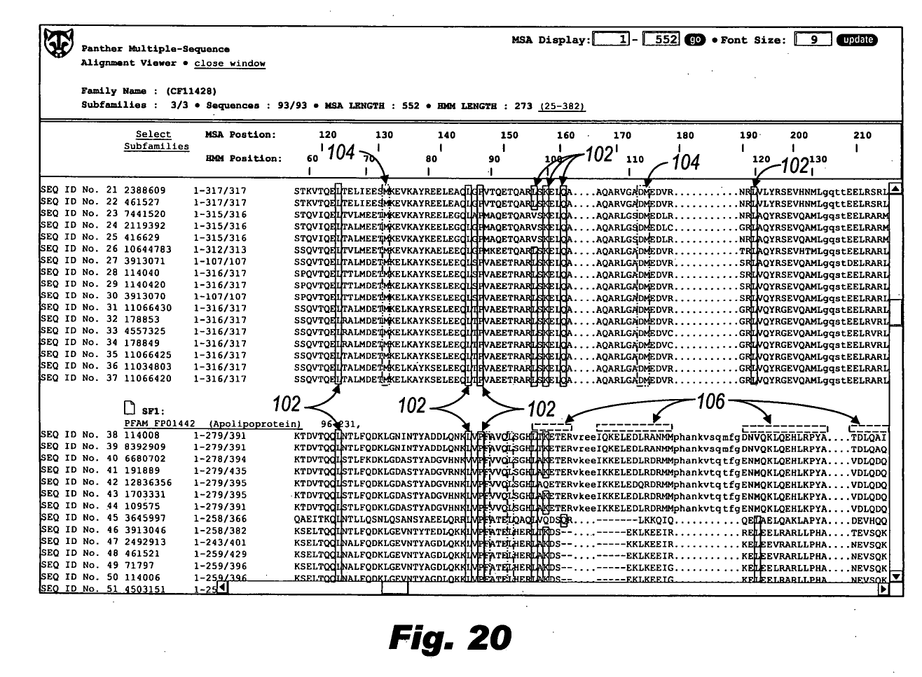

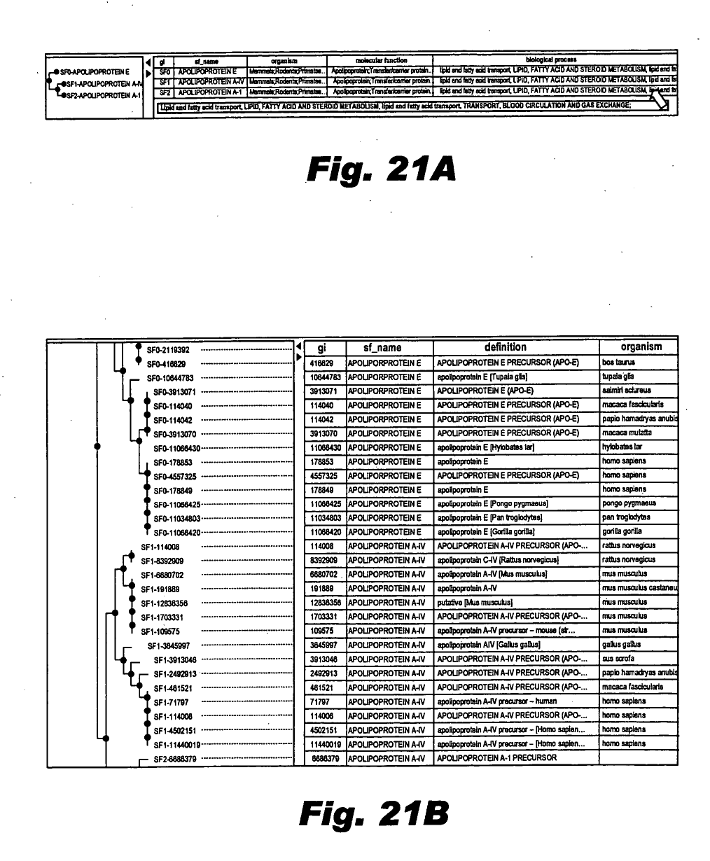

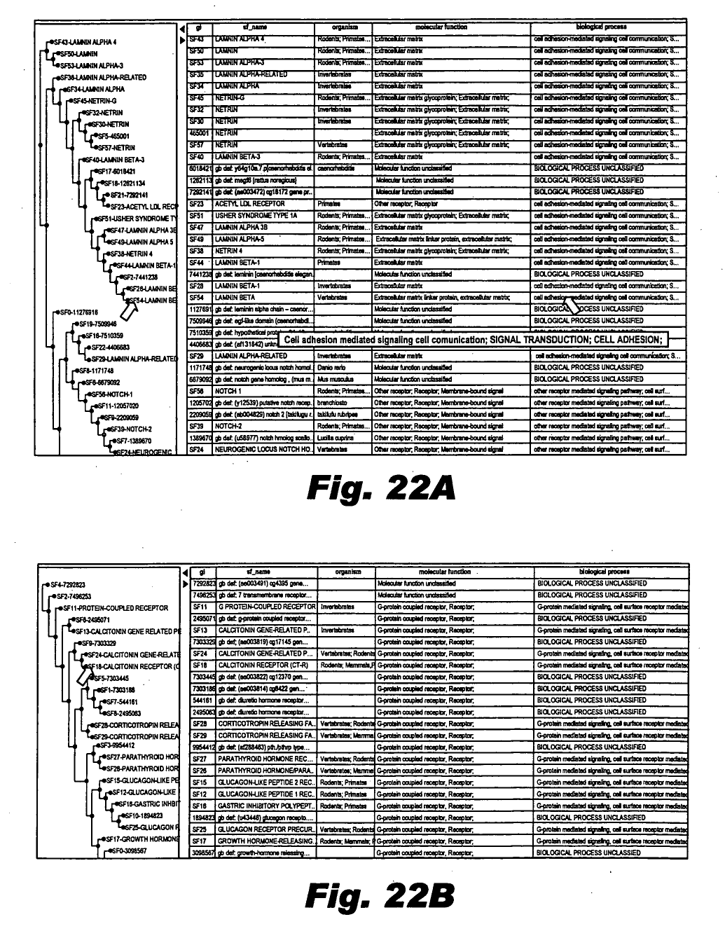

The phylogenetic trees, including the curator-defined subfamily divisions, can be viewed as GIF images. Subfamily nodes can be expanded to view sequence-level annotations from GenBank and SWISS-PROT, to verify curator definitions as illustrated in FIGS. 21 and 22. More specifically, FIG. 21 illustrates the browsable database tree-attribute view for verifying curation including: (A) the “collapsed view”, showing the curator-defined subfamilies and ontology associations in FIG. 21A; and (B) the “expanded view”, showing all of the constituent sequences and their annotations in FIG. 21B. Forms may also be provided to make it easy for users of the browsable database to help correct names and ontology associations, and keep them up-to-date.

Accurate assignment of function using HMMs from curated protein families and subfamilies may be accomplished by curators. The index functional ontology associations for gene products have been shown to be very accurate, primarily due to the emphasis on biologist curation, and to the tree-based homology inference method. Accordingly, curators may define subfamilies in the context of a phylogenetic tree. Trees may be constructed for each family to represent the sequence-level relationships. A biologist curator may then review the tree, dividing it into subtrees (subfamilies) such that all the sequences in a given subfamily can be given the same name and functional assignments. Names may be free-text, while the functional assignments may use controlled index ontology terms. The family and subfamily groupings can provide sets of training sequences for building HMMs.

The design of the browsable database, and the curation effort in particular, may be biased toward functional annotation and ontology classification. Most of the curation effort can be devoted to assigning functions in the context of a phylogenetic tree representation, using functional information from SWISS-PROT and GenBank records, as well as more detailed information, if necessary, in OMIM and PubMed abstracts. A browsable database family may be defined to be as diverse as possible (increasing the number of sequences from which functional inferences can be made) while keeping it tight enough that the resulting tree may be accurate. In some embodiments, alignments or trees may not be hand-curated, and families may not even be mutually exclusive; instead, curators may judge them on how well they perform functional annotation. The tree-building algorithm may be based on a distance metric derived from HMM scoring, so if proteins with the same function can be located in the same subtree, the resulting subfamily HMMs can be predictive of function.

Competition between family and subfamily-level HMMs allows appropriate homology-based inference. The family and subfamily HMMs may then be used to score sequences that were not in the training set. One of the advantages of the browsable database may be the ability to assign specific functions, without overgeneralization. A sequence database search may commonly assign function based on the best hit. The advantage may be that this assignment can be very specific, such as a GPCR having serotonin as a ligand. The disadvantage may be that it can be difficult to know when the query may be too distant from the hit, such that the inference of serotonin binding may be therefore incorrect. A family database search, on the other hand, may generally be correct in associating a sequence with a family, but may not capture the specificity of function in divergent families. For example, there can be members of the aldo-keto reductase family that function as ion channel subunits. The browsable database may combine the advantages of both methods by including both family and subfamily models in the HMM library. If the best hit may be a subfamily HMM, then a specific annotation can often be made, while a family HMM best hit often allows a less specific annotation. Following the example above, a family-level best hit may result in the annotation “aldo-keto reductase 2 family member” and no curated ontology terms, while a subfamily hit may result in the annotation “potassium voltage-gated channel, beta subunit (family 6, subfamily A)”, and the ontology associations voltage-gated potassium channel (molecular function) and cation transport (biological process).

In some embodiments, all significant HMM scores may be stored for each FlyBase Drosophila protein, and LocusLink human protein. The classification of each gene product can be based on the best HMM score. For non-experts, whenever an HMM score may be reported, it may be accompanied by a ‘relation’ icon that indicates the relative certainty of the classification. As the scores become less significant, the probability becomes higher that the classification may be in error. Even using a permissive score cutoff of −35 (‘distantly related’, i.e., the lowest degree of certainty), the total error rate for Drosophila molecular function classifications may be less than 2%.

Because the library may include over 40,000 HMMs, it may not yet be practical to provide a general web interface for HMM scoring of user-defined sequences. However, the library HMM scoring can be made available as an additional service, or for collaborations.

The browsable database HMM annotations may differ from domain-based HMM annotation. Databases such as Pfam and SMART have used the HMM formalism to provide an extremely useful tool for identifying conserved functional and structural domains in a protein sequence. The browsable database may use HMMs somewhat differently, with the goal of annotating the overall biological function of a protein. Like Pfam and SMART, the database family-level HMMs often may have a functional annotation based on a single domain. The database subfamily-level HMMs (and many family-level HMMs as well), however, can be more informative than the simple sum of the individual domain annotations. For example, the protein encoded by the human gene HSPG2 contains many different domains, including the LDL receptor A domain, epidermal growth factor repeat-like domains, immunoglobulin-like domains and both laminin B and laminin G domains. Each of these domains may be found in different combinations across a variety of proteins having divergent functions. The only one of these domains that can be assigned a consistent function may be the laminin-type EGF domain, which has been assigned by Interpro to the Gene Ontology (molecular function) term structural molecule. By contrast, the highest scoring HMM of the browsable database may be the subfamily heparin sulfate proteoglycan perlecan (CF10574:SF31), which may be assigned to the index ontology terms (molecular function) extra-cellular matrix glycoprotein, and (biological processes) cell adhesion and cell adhesion-mediated signaling. This can be a specific subfamily of the broader browsable database family laminin-related (CF10574), which, like the Pfam laminin B and G domains, may not be assigned to any functional terms. FIG. 22A illustrates a related example of database subfamilies capturing functional divergence. In particular, laminin-related proteins have divergent domain structures (which correlates with divergence within the shared laminin domain), and this case can be modeled using subfamily HMMs.

Even for single-domain proteins, the browsable database subfamily HMMs often allow for more specific functional inferences than may be possible from more general HMMs, such as Pfam and SMART. For example, the CALCR gene product hits the Pfam HMM for the secretin-like seven transmembrane receptor family, which may be assigned to the GO molecular function G protein-coupled receptor. The highest-scoring HMM of the browsable database may be the subfamily calcitonin receptor (CF12011:SF18), which may be assigned to G protein-coupled receptor, as well as to the biological processes skeletal development and other neuronal activities. The more specific assignments can be correct for this subfamily but not for all members in the larger family. FIG. 22B illustrates a related example of database subfamilies capturing functional divergence. In particular, secretin-related GPCRs have divergent sequences within a common domain, and this case can be modeled using subfamily HMMs.

As described above, the browsable database can be a system for classifying and predicting the functions of protein sequences in the context of sequence-level relationships. The browsable database may define a controlled vocabulary for protein annotation, as well as a method for classifying new sequences. The process by which users employ the browsable database to find genes by browsable database families protein classification may be described in greater detail below.

According to some embodiments, users can employ a browser. The browser may allow users to: (1) browse functional categories and protein families/subfamilies; (2) text search functional categories or protein families/subfamilies; (3) create a gene list; (4) view a philogenetic tree for a given family; (4) view the a multiple sequence alignment for a given family; and (5) view the database “partial” multiple sequence alignment for a given family.

The gene list that appears when users browse or text search protein classification data of the browsable database may differ from a gene list that appears when they search other data sources. More information may be provided below about the gene list.

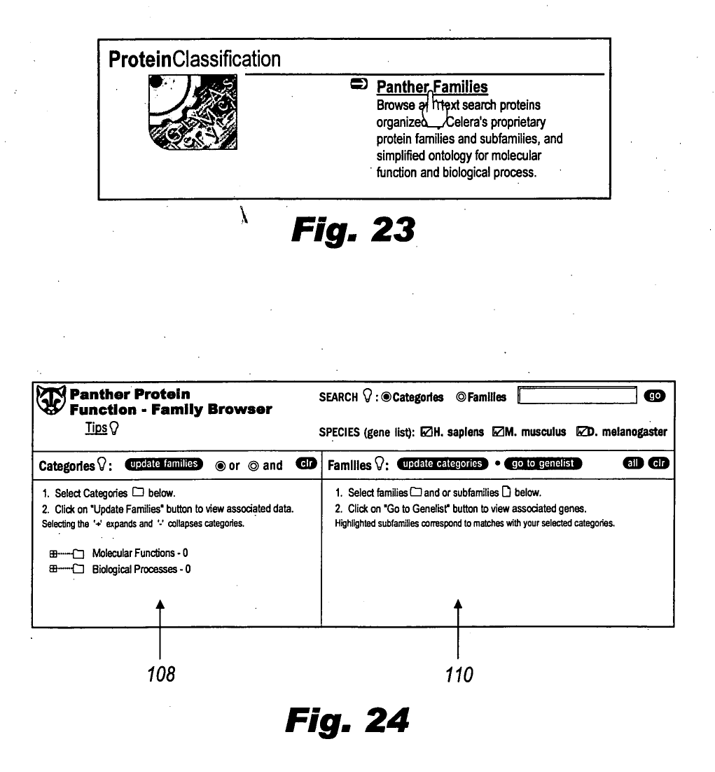

When browsing functional categories and protein families/subfamilies, users can perform the following steps. From a library page, users can select a families button as illustrated in FIG. 23. Then, the browser may appear as illustrated in FIG. 24. User can browse proteins first by functional categories, and then by family and subfamily. The browser may display the mapping of protein functions in left panel 108 to protein families and subfamilies in right panel 110.

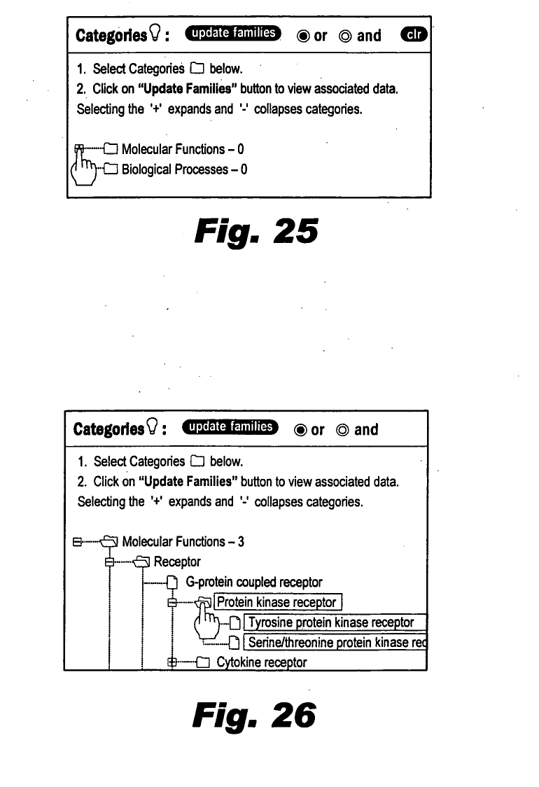

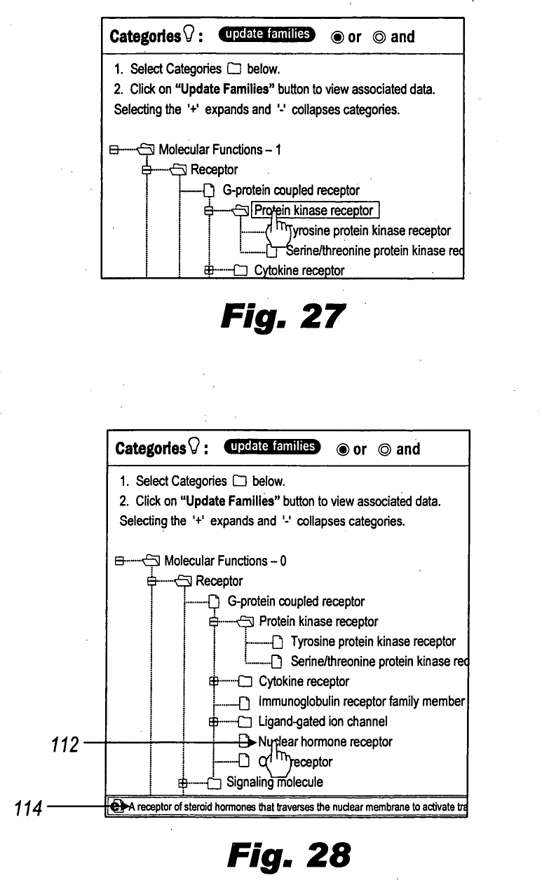

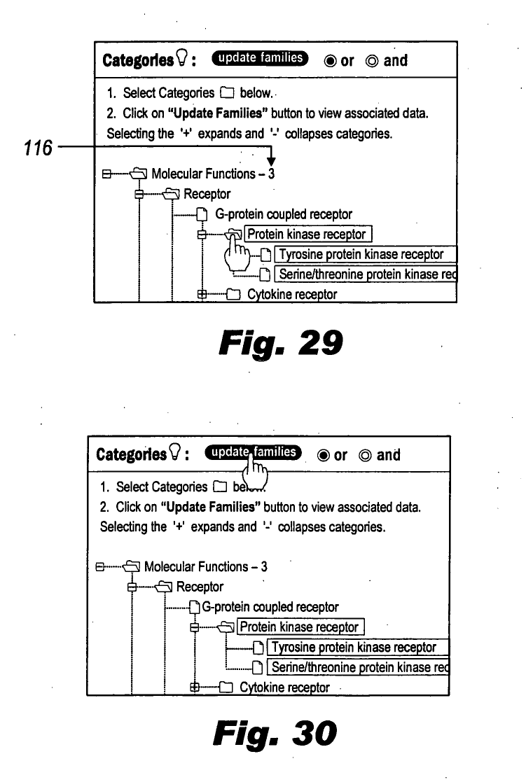

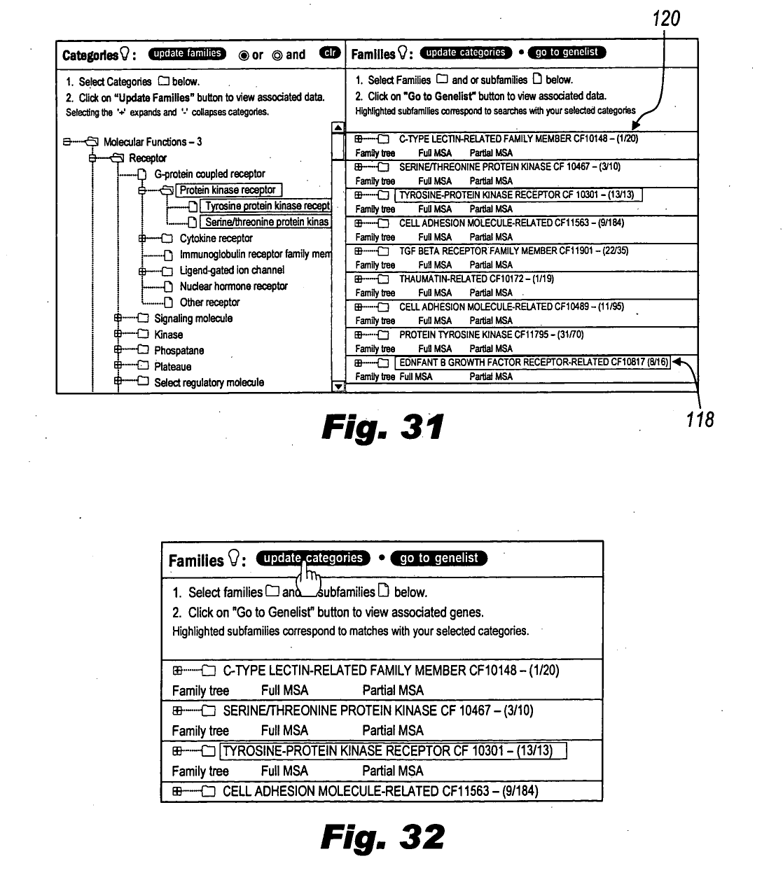

The navigation can be based on a file-folder analogy. For example, users can click the ‘+’ next to a folder to view children of a parent category as illustrated in FIG. 25. Then, users can click a folder to select the parent and all of its children as illustrated in FIG. 26. Alternatively, users can click on the category name to select only that category as illustrated in FIG. 27. As illustrated in FIG. 28, users can mouse over a category as at 112 to view the definition of a given category at the bottom of the browser window as at 114. FIG. 29 illustrates that the browser may display the total number of different categories selected in each ontology (including all selected children) next to each ontology heading (molecular function or biological process) as at 116.

After users select a set of categories, they can click a radio button to specify a Boolean operator governing how to retrieve the database families/subfamilies assigned to those categories as illustrated in FIG. 30. A default “or” operator may be essentially a “set-union” operation over the selected categories: “all families/subfamilies whose members can be assigned to protease OR developmental processes.” An “and” operator may be a “set-intersection” operation over the selected categories: “all families/subfamilies whose members can be assigned to protease AND to developemental processes.” If users click the and radio button, they may need to take care to select only a category by clicking on the name and not the folder, since the children can be often mutually exclusive and the selection may not have the desired result. For example, if users select protease and all of its children by clicking the folder instead of the name, this may imply: “all families whose members can be assigned to protease AND serine protease AND cysteine proteases, because each of these catalytic mechanisms may be exclusive of the others.

Once users have selected a set of functions and an operation (and/or), they can click “update families” to retrieve protein families/subfamilies that match the selections as illustrated in FIG. 30. The browser may display these families/subfamilies in the right panel. The families and subfamilies that may have been assigned by expert curation may be highlighted by default as at 118. Since not all subfamilies in a given family may share the selected function(s), not all family/subfamily names may be selected. The browser may display the number of selected subfamilies and total subfamilies next to the family name as at 120. Users can modify the default selections in the Families panel by selecting/deselecting various families/subfamilies.

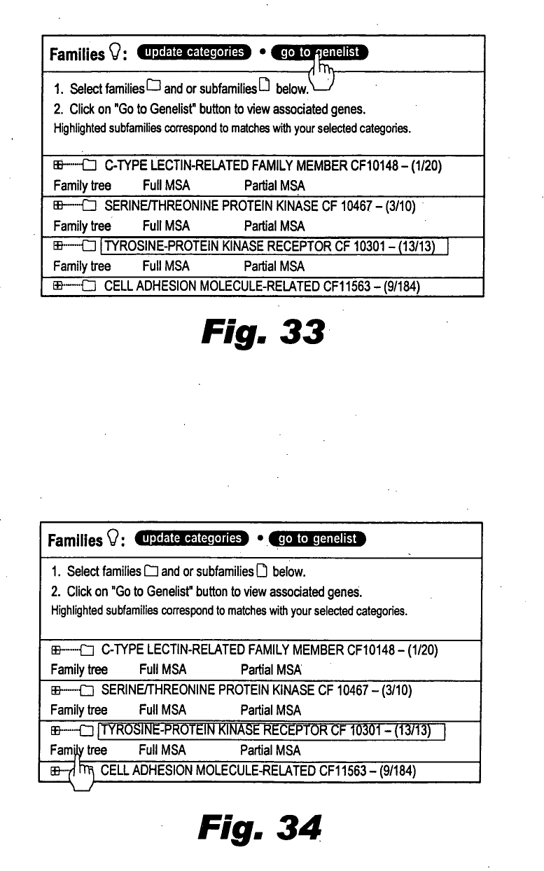

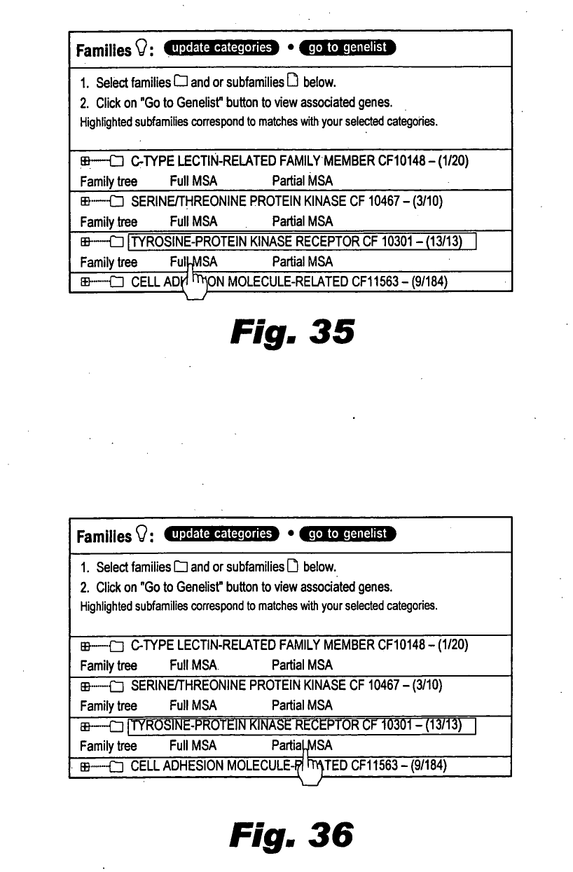

As in the Category panel, users can click a folder to select a parent and all its children, or click a name to select only the parent. From the Families panel, users can view all functional categories for selected families/subfamilies. They can also click “update categories” to highlight in the left panel all functional categories to which the selected families and subfamilies have been assigned as illustrated in FIG. 32. Clicking “update categories” may cause previous selections in the left panel to be lost. Users can further create a Gene list by clicking “go to genelist” to open the gene list for all proteins assigned to all selected families/subfamilies as illustrated in FIG. 33. As mentioned above, the gene list that appears when users browse or text search browsable database protein classification data may differ from the gene list that appears when users search other data sources. Yet further, users can view the browsable database tree for a given family by clicking the “Family Tree” hyperlink that appears under the family name's folder as illustrated in FIG. 34. Further still, users can view a database Multiple Sequence Alignment for a given family by clicking the “Full MSA” hyperlink that appears under the family name's folder as illustrated in FIG. 35. Finally, users can view the browsable database's “partial” MSA for only selected subfamilies of a given family by clicking the “Partial MSA” hyperlink that appears under the family name's folder as illustrated in FIG. 36.

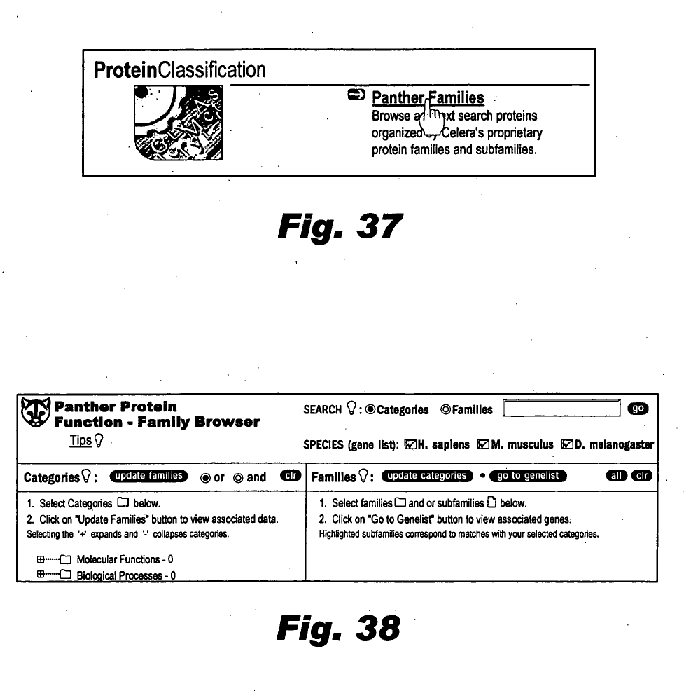

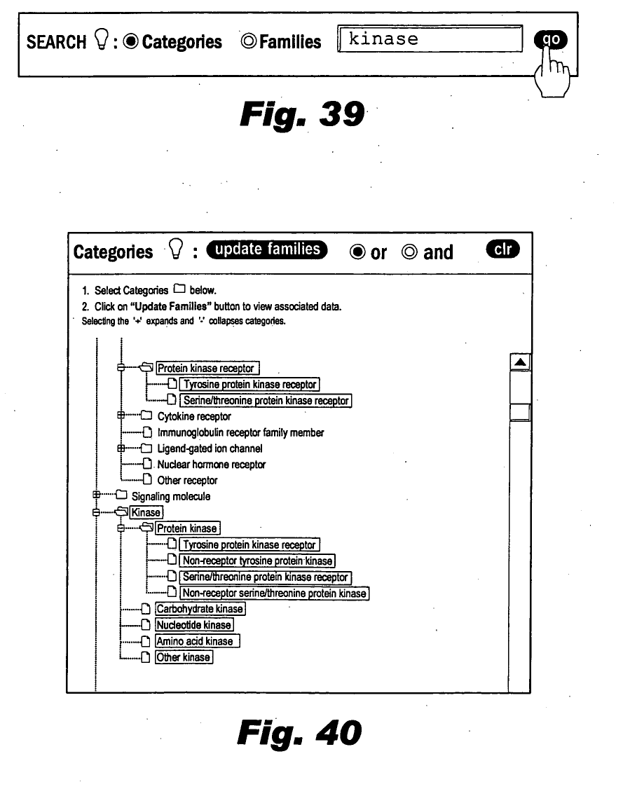

In addition to browsing, users can also text search against functional categories. For example, users can start by clicking a families button from library page as illustrated in FIG. 37. The browser may then appear as illustrated in FIG. 38. Next, users can click the “Categories Search” radio button, and next type a search string in the text box. For example, users can type “kinase” and then click go as illustrated in FIG. 39. This action may open the folders in the browser's left panel appropriately, such that all categories that contain the search term in the category name can be visible and highlighted as illustrated in FIG. 40. From this point, users can browse functional categories and then protein familes/subfamilies to refine results.

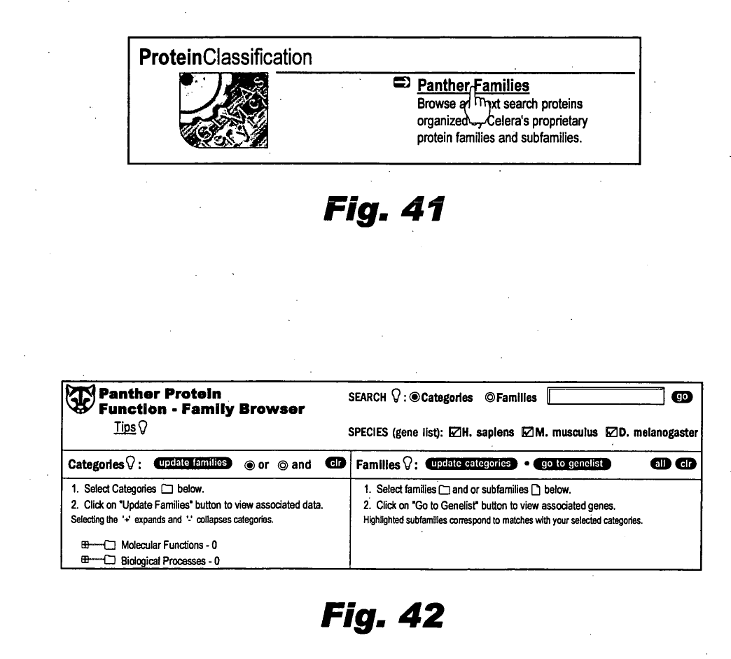

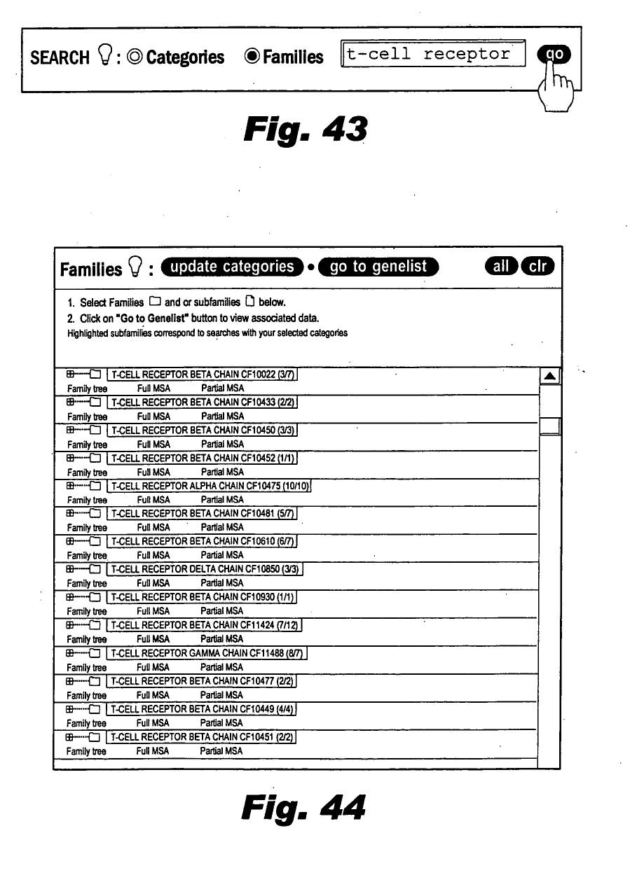

Users can further text search against protein families and subfamilies. Starting at the library page, users can click a families button as illustrated in FIG. 41. Next, the browser may appear as illustrated in FIG. 42. Then, users can click the “Families Search” radio button and type a search string in the text box. For example, users can type “t-cell receptor” and then click “go” as illustrated in FIG. 43. This action may retrieve all families for which either the family or subfamily name (or both) contain the search term. The browser may display these families and subfamilies in the right panel, with the appropriate names highlighted as illustrated in FIG. 44. From this point, users can browse protein families/subfamilies and functional categories to refine results.

Users can create a gene list by browsing or text searching to select the desired protein families/subfamilies in the Families panel as described above. Users can select family and subfamily assignments independently. When users select a family name only (by clicking on the text of the name), the gene list will contain proteins assigned to that family, but not any proteins assigned to specific subfamilies. When users select a subfamily name, the gene list can contain proteins assigned to that subfamily.

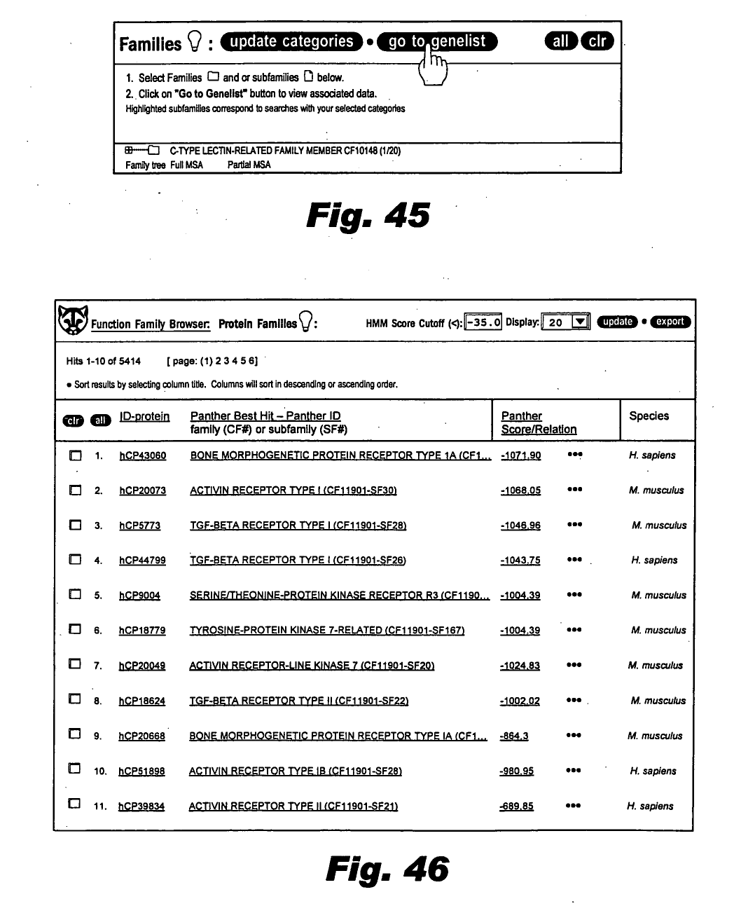

If desired, users can select or deselect Species checkboxes to specify which Genome(s) to search to create the gene list, and then click “go to gene list” as illustrated in FIG. 45. The gene list may appear in a new window as illustrated in FIG. 46. This window can list all proteins assigned to the selected families/subfamilies. All protein sequences may have been scored against a full library potentially containing over 2200 family-level and almost 40,000 subfamily HMMs, and may be assigned to the family or subfamily model having the best HMM score.

As a result of scoring, the models can distinguish between sequences that most likely belong to an existing subfamily, and sequences that can be most likely part of a novel subfamily (or a subfamily not represented in the library). Family-level models and subfamily level models can be generally assigned quite differently to functional categories, since a more detailed functional prediction can often be made for close, subfamily-level relationships.

The gene list allows users to perform several actions. For example, users can sort the list by clicking on any of the underlined column names as detailed in Table 2.

TABLE 2

| Column | Description |
|---|---|
| ID-Protein | Protein ID. |
| | The Protein IDs in this column can be |
| | hyperlinks to the corresponding |
| | BioMolecule Report. |
| Best Hit | Name of the best-scoring HMM, The |
| | best hits in this column can be |
| | hyperlinks to the corresponding |
| | family/subfamily in the browser. |
| ID | ID of the best-scoring HMM |
| Score/Relation | HMM score. |
| | The HMM scores can be hyperlinks to |
| | the HMM alignment. |

By default, the list may be sorted by HMM score, which may be a quantitative indicator of how confident the functional assignment may be (“more negative” scores can be higher confidence). Users can also sort by best-scoring HMM ID. This option may cluster proteins in the same family/subfamily together, thereby grouping possible orthologs/paralogs. Users can also modify the list to exclude lower-confidence predictions using the HMM Score Cutoff textbook at the top of the list. The weakest score stored in the database may be −20. It may be helpful to have a cutoff of “−35” to get a list of proteins that can be very likely to be correctly assigned to a given protein family or molecular function, and a cutoff of “−85” for very high confidence assignments of molecular functions and biological processes. Users can further export the list to save it to local disk in a tab-delimited format.

Users can also access the browsable database tree viewer. Distance trees may allow users to explore the relationships between sequences in a particular family, as well as view some of the key information used to annotate the families and subfamilies. In some embodiments, the trees can contain only publicly-available protein sequences (SwissProt and GenPept). Various display conventions may be employed to represent tree elements of different types. In some embodiments, blue diamonds can represent subfamily nodes. Subfamilies may be colored to help distinguish between different subfamilies. Aside from this, the subfamily color may not have any special significance.

The tree viewer has two panels that can be mapped to each other. The first panel displays the relationship between the different sequences. The longer the (horizontal) branch length, the more distant may be the groups joined by those branches. Vertical branch length may be fixed for ease of viewing together with the second panel, the “attribute table.” The attribute table can contain one row for each sequence in the tree. Each column may display a different attribute of the sequences. For example, a “gi” column can provide the GenBank accession number for the sequence. Clicking on the accession number may open the full SwissProt record if the sequence has been reviewed by SwissProt, or the full GenPept record if it has not been reviewed by SwissProt. Also, a “definition” column may provide the brief definition line parsed out from either the SwissProt (whenever available) or GenBank record to allow users to scan the sequence-level annotations. Further, an “organism” column may provide the organism from which the sequence was derived. Clicking on the organism name can open the full taxonomy record for that organism. Further, an “xlinks” column may provide hyperlinks to relevant abstracts from PubMed.

This page may also linksto the multiple sequence alignment view directly. Users can view a “Full” Multiple Sequence Alignment for a given family by clicking the “Full MSA” hyperlink. Alternatively, users can view a “Partial” MSA for only selected subfamilies of a given family by clicking the “Partial MSA” hyperlink.

The tree viewer may also highlight selected subfamilies. These can be indicated by red bars on the left-hand side of the tree. Users can modify the list of selected subfamilies by clicking the “Select subfamilies” hyperlink. If users launched the tree viewer from the browser, it may highlight all of the subfamilies selected in that viewer. If users launched the Tree Viewer from the MSA Viewer, the appropriate subfamily may be highlighted.

The tree viewer can support two views. For example, the collapsed view may provides a high-level view of the tree, in which subfamilies may be the most specific “leaves” of the tree. The subfamily name given by curators may appear in the “gi” column of the collapsed view. The range of species found in each subfamily may be summarized in the “organism” column. In some embodiments, this organism summary can be made using a mapping file from GenBank that unfortunately classifies fungi as “plants.” In other embodiments, this known bug may be fixed. Also, the expanded view can contain the full tree, complete with sequence-level annotations and hyperlinks.

Users can toggle between the expanded and collapsed views in two different ways. For example, when the tree may be collapsed, users can click on the “Display expanded view” hyperlink just above the tree panels. Also, when the tree may be expanded, users can click on the “Display collapsed view” hyperlink. Clicking on these hyperlinks may not change the subfamily selection. Clicking on a subfamily node in the tree may change the subfamily selection to the selected subfamily in addition to collapsing or expanding the tree. Users can also change subfamily selections by clicking on the “Select Subfamilies” hyperlink just above the tree panels. Then, users can select or deselect subfamilies by clicking on the checkboxes, followed by clicking “go”.

Users can further access the browsable database's multiple sequence alignment viewer. Multiple sequence alignments (MSAs) may serve as the basis for the distance trees, and therefore of the family/subfamily classification. Users can view them in two modes. For example, the full MSA mode may include all (publicly available) sequences in the family that can be related closely enough to produce an informative multiple alignment (i.e., the resulting trees and HMMs can be useful for function prediction at both a family and subfamily level). Also, the partial MSA mode can show the alignment for only the currently selected subfamilies.

In the MSA viewer, users can perform several actions. For example, users can change the selection of subfamilies shown by clicking on “Subfamily Selection”, just as in the tree viewer. Users can also focus on only a part of the sequence alignment (“range”). Users can further change the font size of the alignment, and jump to the start or end positions of the HMM alignment (by clicking on the links after the HMM length). The MSA view may be divided into subfamilies in the same ordering as in the tree. In this way, the most closely related sequences may appear closest to each other in the alignment.

In the MSA viewer, there can be two panels, an information panel on the left, and an MSA panel on the right. The left panel may contain information about each subfamily and sequence. Each of these subfamilies and sequences may also be hyperlinked to more detailed information. For example, users can mouse over a subfamily number (SF) to see the subfamily name, and click on an icon to the left of the subfamily number to open the browser with the selected subfamily loaded and highlighted in the right panel. Also, users can click the “Tree” hyperlink to open the browsable database tree for the appropriate family, with the selected family highlighted. Further, GenBank accession numbers and the range of the sequence that may be aligned to the HMM can be accessed by clicking the accession numbers to open the corresponding Swissprot or GenBank records.

The right panel can display the multi-sequence alignment, which may be generated by aligning the sequences to the family HMM. The alignment can be in the conventional HMM format. The MSA may be numbered according to both the position in the overall MSA and the position in the HMM. Users can employ the horizontal scroll bar on the bottom to see the entire alignment. The MSA viewer may use three colors to describe positions in the alignment. For example, red can signify subfamily-specific conservation by denoting a column that may be 100% conserved within a subfamily, but the same amino acid does not appear in that position in any of the other subfamilies. Also, black may signify globally highly conserved by denoting a column that may be >90% conserved across the entire alignment. Conservation may calculated after appropriate weighting of sequences so that a large subset of closely related sequences does not skew it. Further, grey can signify globally moderately conserved by denoting the same as for black positions, except that the conservation may be between 75% and 90%. The choice of color scheme may vary in some embodiments.

Users yet further may access the browsable database's HMM alignment view. This view may show the query sequence aligned to the consensus sequence for the HMM (can be either a family or subfamily HMM). The alignment format can follow the HMMer conventions. For example, the top line may be the HMM consensus—i.e. each position may be represented by the most probable amino acid for that position. An upper-case letter can indicate that the residue shown may be highly conserved (probability>0.5). A dash may only appear in subfamily HMMs, and can indicate where the subfamily has a deletion relative to the family. A period (‘.’) can represent positions where the query sequence has an insertion relative to the HMM. Also, the bottom line may be the aligned sequence, and the format can follow the conventional HMM format. Thus, for amino acids modeled by the HMM, upper-case letters can be “matches” and indicate positions where the amino acid scores well against the HMM. Dashes may denote positions where a particular sequence has a deletion relative to the HMM. For amino acids not modeled by the HMM, lower-case letters can be “inserts” relative to the HMM. These amino acids may be shown only so the entire sequence can be viewed. A column that may be not modeled by the HMM may only contain periods and lower-case letters, such that these columns should not be interpreted as part of the multiple sequence alignment. Further, the middle line can indicate the level of “matching” between the HMM consensus and the aligned sequence. An amino acid letter may indicate that the sequence matches the consensus at a given position. A “+” can indicate that the aligned amino acid has a better score than background, i.e., that it scores well against the HMM even if it does not perfectly match the consensus.

Users can still further access an “all family/subfamily hits view” of the browsable database. This page may show all of the family/subfamily HMMs that hit a query sequence (with a score better than a certain threshold). Family HMM hits may be shown if the score may be better than 020, and subfamily HMM hits may be shown if the score may be better than −35. The page can be arranged such that all hits in a given family can be grouped together, best scores first. Users can view alignments by clicking on the score, and can view a protein family or subfamily in the browser by clicking on the family/subfamily name. In some embodiments, the system may display scores only if the score may be better than −35, and displays only the top-scoring HMM and associated information for a protein.

Another embodiment of the of the protein classification system may be described below with an emphasis on the ability of the system to infer biological function. In particular, the system can infer the function of uncharacterized proteins, predict biological role for pathway building, and enhance interpretation of expression information.

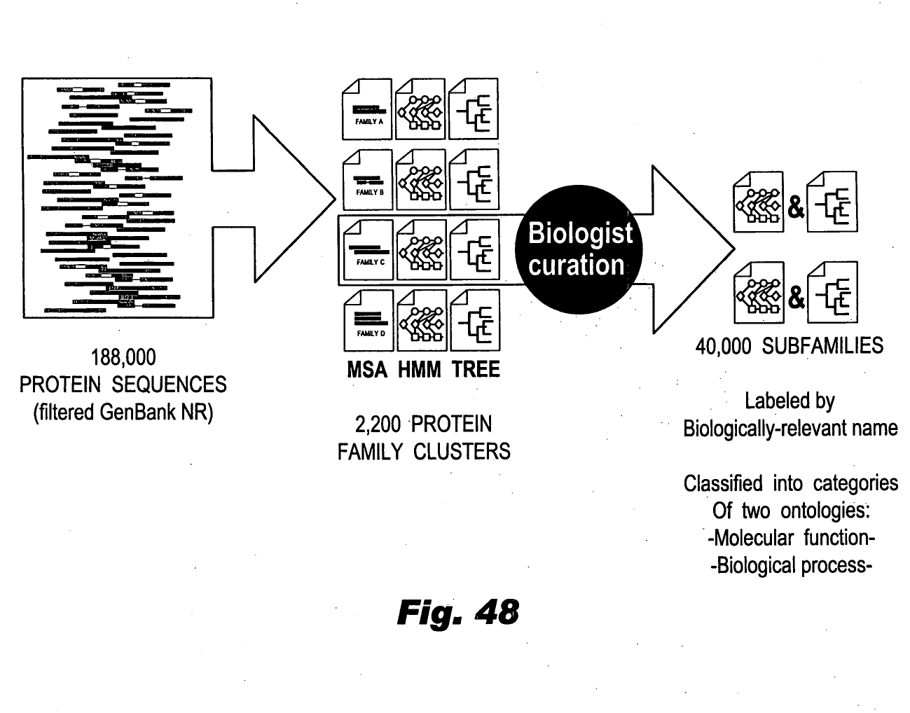

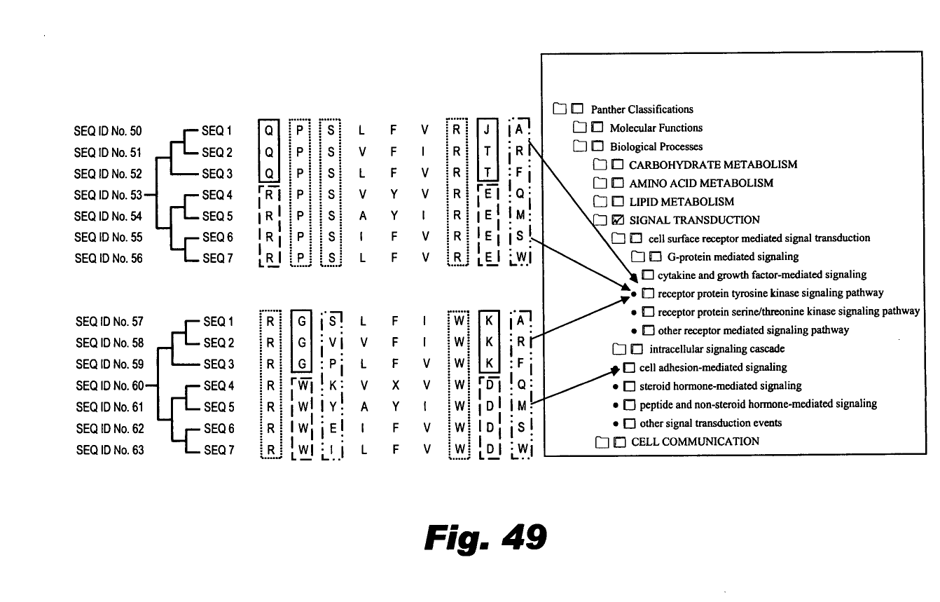

The browsable database's proprietary protein classification system can provide researchers with an understanding of protein function for known and novel human, mouse and Drosophila proteins. The browsable database may have many advantages over current protein classification systems because it can use both a statistical modeling approach and specific protein annotation information to define families and subfamilies of proteins. A three-stage process may be employed to build the browsable database. First, all of the known proteins may be clustered into families based on global sequence similarity. Biologists can then define a controlled vocabulary for protein annotation and refine the library families further into subfamilies by breaking each family into groups of sequences that have common molecular function(s) and participate in common biological processes. Each subfamily may also be given a name using controlled vocabulary. This process can generate statistical models for all predefined families and subfamilies as shown in FIG. 48, which may then be applied to the proteins in Human, Mouse and Drosophila Assembled and Annotated Genomes, allowing inference of both molecular function and biological processes. These results can be presented in the in an intuitive, easy-to-use interface. This knowledge aids in the identification of the potential function of novel proteins and better interpretation of biological response-based studies such as differential expression and array based gene expression experiments.

A method for constructing a browsable database for use with biological information may start with clustering of protein sequences into families. The library may be constructed by first clustering full-length proteins of many species (eukaryotic, prokaryotic and viral proteins) from the GenBank NR database into families, requiring that all members of a family have aligned regions that span a majority of the total sequence length. This clustering can result in a partitioning of protein space into groups of proteins that share homology across their entire length.

In the browsable database, a family can be defined as a group of sequences for which a high-quality multiple sequence alignment can be generated. This capability may be helpful for building a “distance tree,” as well as for analyzing the multiple alignment for conserved and variant positions as a function of subfamily relationships. A number of numerical measures may be employed to automatically assess alignment quality, in addition to expert assessment of the resulting distance trees. If an alignment fails any of these measures, the family may be made still more restrictive.

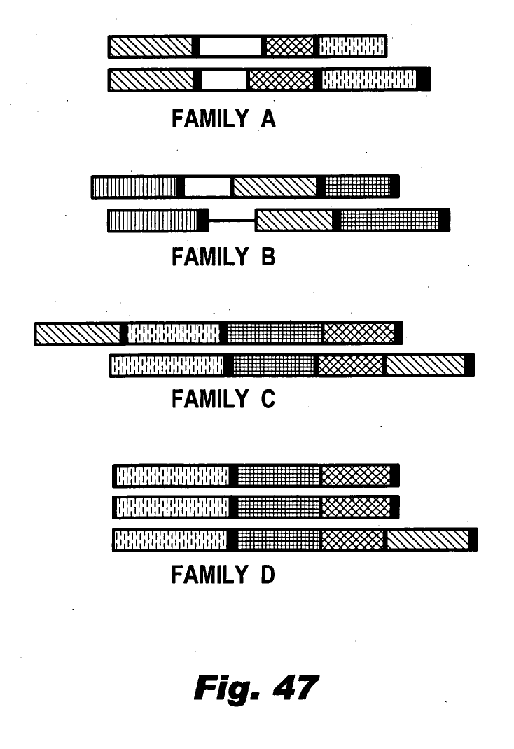

FIG. 47 provides a schematic representation of the organization of proteins into families by multiple domains. The members of the families have aligned regions that may span a majority of the total sequence length. This alignment can result in a partitioning of protein space by groups of proteins that share homology across their entire length and not just one domain.

FIG. 47 illustrates clustering all of the known proteins into families based on a global sequence similarity. Biologists can then define a controlled vocabulary for protein annotation and divide each of the library families into subfamilies (subtrees) using information about shared molecular function(s), and participation in common biological processes. This process can generate statistical models for all defined families and subfamilies (such as about 52,000) that can then be applied to all the proteins in the Assembled and Annotated Genomes, allowing inference of both molecular function and biological processes.

Once proteins can be grouped into families based on their global domain organization, the families can be aligned using Hidden Markov Model methods (HMM). The resulting HMM may then be used to “extend” the original family to include additional members that have strong local matches. In this way, sequence fragments can be included, as well as proteins that may match only over a single domain. Family trees may then be produced from these high quality robust alignments, and the trees can then be reviewed. As long as the trees can be divided into subtrees of proteins with conserved function, then the subfamilies may be useful for function prediction even if some of the alignments span only a single domain.

The method of construction may then proceed with biologist curation and subfamily classification. Each protein family may be reviewed and annotated by a team of expert curators. Unlike other reported approaches toward curation, browsable database construction process's curation may be performed in the context of a “distance tree”: i.e. a family of sequences may be annotated in the context of the set of (nearly) all related proteins. This context can allow curators to make inferences that could not be made if they were looking at a single sequence at a time, as well as perform consistency checks on the incoming data and the annotations they make themselves. The curators of the families may be selected based on areas of expertise.

The annotation process can have a carefully defined protocol. A protein family distance tree may be linked to sequence-level annotations for each sequence in the family (derived from the GenBank NR database). Curators can also use links to more detailed external information, including PubMed abstracts. Information about the curation process may be recorded for each family, including the name of the annotator, the date of annotation, and any problems or outstanding issues uncovered during curation as a quality control step.

The concept of “subfamily” may be helpful to understanding the true value of the browsable database. A subfamily may be defined as a subtree of the family tree, all of whose sequences share an “attribute” in common. In the browsable database, an arbitrary number of attributes may be used to divide the tree into subfamilies. In the library, the attributes used to define subfamilies may be nomenclature (often related to molecular function), molecular function category and biological process category. Because subfamilies can also be subtrees of a “distance tree” (where the distance may be defined in terms of HMM scores), each subfamily may be represented by an HMM that can be compared to other subfamilies to reveal the sequence-level determinants of functional specificity. The benefit of this subfamily organization may be that proteins that not only share general biological function (as defined by their family association), but also subdomain specificities, can be truly closely related with regard to their biological roles. For example, in the biogenic amine family, histamine H2 receptors can be a distinct subfamily from serotonin HT1A, and these ligand-binding differences can be related to amino-acid level differences between these subfamilies.

In addition to reviewing the membership of sequences within a subfamily, the expert biologists can give each subfamily a biologically meaningful name. Not all sequences must be individually annotated in exactly the same way for the curator to decide that they all, in fact, can be likely to share the same attributes. In fact, the lack of standards for nomenclature, the wide range of annotation quality and the years of transitive sequence annotation have made biologist interpretation advantageous. The expert's ability to infer the functions of proteins that can be either incorrectly or inadequately annotated may therefore be captured by the browsable database.

Another component of the browsable database may be the ontology, or index, for molecular functions and biological processes. Each family and subfamily may be assigned individually to the appropriate function and process categories as illustrated. In particular, FIG. 49 illustrates assignment of subfamilies to biological process and molecular function categories. Subfamilies may be defined as subtrees of a “distance tree” representing a protein family. Sometimes, entire families can be assigned to a category, but most often, subfamilies may be individually assigned to categories with greater specificity. The index may be developed with reference to the publicly-available Gene Ontology (GO; Ashburner et al., 2000). However, compared to GO1, the index may be greatly simplified (only about 250 categories under molecular function arranged into three levels, compared to over 7000 categories in GO up to 12 levels deep) to facilitate browsing and high-level analysis of large gene sets. The index may also contain several mammalian-relevant categories, such as acquired immunity or developmental functions, that can be currently missing from GO.

The method of construction can further include assigning proteins to families and subfamilies as illustrated in FIG. 49. Predicted proteins from genomes (currently human, mouse and Drosophila) may be scored against the library of, for example, 6155 family-level and 52,000 subfamily-level HMMs. Each predicted protein can be annotated with the name, molecular functions and biological process of the highest-scoring HMM. The advantage of this approach may be that, unlike BLAST-based functional assignment, new proteins can be annotated differently in the case of family-level versus subfamily level similarity. This can often prevent over-interpretation of sequence similarity results.

In conclusion, the browsable database can offer a specific, sensitive and accurate categorization of proteins into categories that may be predictive for their molecular function as well as their biological roles. Using the library, which can contains over 210,907 training sequences organized into 6155 families and 52,000 subfamilies that span wide evolutionary distance, users can leverage the benefit of all identified human, mouse and Drosophila proteins having been accurately placed in their appropriate families and subfamilies. Assignment of these subfamilies to specific biological processes and molecular functions can facilitate the identification of relevant pathways that participate in diseases of interest to investigators and the identification of novel targets, their functional homologs, and therefore improved target prioritization. Organization of all human, mouse and Drosophila proteins into subfamilies can also facilitate the identification of homologs that can have significant impact on target prioritization. For example, knowledge of all close homologs to a putative target can influence the design of optimally specific small molecules or monoclonal antibodies that minimally react with these homologs, thus minimizing the unwanted side-effects. The benefit of this organization can be the ability to better prioritize which targets to pursue based on the likelihood that cross reactivity will create downstream complications in drug specificity.

There can be many uses for the system constructed according to the preceding method For example, users can browse the proteins predicted by the human, mouse and Drosophila genomes. Also, users can create gene lists for aiding analysis of mRNA and/or protein expression results. Expression-based clusters can be correlated with biological processes, or gene products of certain target classes can be identified (Cho et al., 2001: Cho & Campbell, 2000). Further, the system facilitates comparative genomics analysis. Predicated proteins from different organisms can be compared by family/subfamily relationships (orthology and paralogy) and by functions and processes. Yet further, the database can allow users to explore protein family/subfamily relationships in the library of phylogenetic trees. Further still, the database can allow users to explore amino acid-level determinants of function and specificity. Finally, the library of multiple sequence alignments can highlight positions that can be conserved across an entire family as well as subfamily-specific positions.

Those skilled in the art can now appreciate from the foregoing description that the broad teachings herein described can be implemented in a variety of forms. Therefore, while various embodiments have been described in connection with particular examples thereof, the true scope of the related teachings should not be so limited since other modifications will become apparent to the skilled practitioner upon a study of the drawings, the specification and the following claims.

1. A browsable database system for use with biological information, comprising;
at least one datastore of biological sequence data, including at least one of gene sequence data and protein sequence data;
an ontology of categories of biological functions mapped to statistical models trained on families of biological sequences related to the biological functions;
an input receptive of at least one user selection indicating a biological function of said ontology;
a recognizer adapted to identify multiple alignments of biological sequence data based on said sequence datastore and a statistical model related to a function indicated by the user selection; and
an output adapted to communicate the multiple alignments to a user providing the user selection.

2. The system of claim 1, further comprising at least one datastore of curated philogenetic trees organized into families of sequences based on global sequence similarity, wherein the families are divided into subfamilies according to sequence function and the families and subfamilies are mapped to appropriate statistical models.

3. The system of claim 2, further comprising an output communicating contents of the philogenetic trees to the user in accordance with user navigation selections.

4. The system of claim 2, further comprising a text searcher receptive of user defined text and adapted to select families and subfamilies of the philogenetic trees by matching the text to contents of the philogenetic trees.

5. The system of claim 2, further comprising an input receptive of a user-defined sequence, wherein said recognizer is adapted to select families and subfamilies related to statistical models achieving high scores respective of the user-defined sequence.

6. The system of claim 1, further comprising an output communicating contents of the ontology to the user in accordance with user navigation selections.

7. The system of claim 1, further comprising a text searcher receptive of user defined text and adapted to select functional categories and subcategories by matching the text to contents of the ontology.

8. The system of claim 1, further comprising an input receptive of a user-defined sequence, wherein said recognizer is adapted to select functional categories and subcategories related to statistical models achieving high scores respective of the user-defined sequence.

9. The system of claim 1, further comprising an input receptive of database selections, wherein said recognizer is adapted to identify sequences in a subset of multiple sequence datastores based on the database selections.

10. The system of claim 1, further comprising an input receptive of a user selection of a Boolean operator, wherein said recognizer is adapted to identify the multiple alignments in accordance with the Boolean operator.

11. A method of operation for use with a browsable biological database, comprising;
communicating an ontology of categories of biological functions to a user, wherein the biological functions are mapped to statistical models trained on families of biological sequences related to the biological functions;
receiving at least one user selection indicating a biological function of the ontology;
accessing at least one sequence datastore of biological sequence data, including at least one of gene sequence data and protein sequence data;
employing pattern recognition to identify multiple alignments of biological sequence data based on contents of the sequence datastore and a statistical model related to a function indicated by the user selection; and
communicating the multiple alignments to the user providing the user selection.

12. The method of claim 11, further comprising communicating at least one set of curated philogenetic trees to a user, wherein the trees are organized into families of sequences based on global sequence similarity, the families are divided into subfamilies according to sequence function, and the families and subfamilies are mapped to appropriate statistical models.

13. The method of claim 12, further comprising navigating and selecting contents of the philogenetic trees in accordance with user navigation selections.

14. The method of claim 12, further comprising:
receiving user defined text; and
selecting families and subfamilies of the philogenetic trees by matching the text to contents of the philogenetic trees.

15. The method of claim 12, further comprising:
receiving a user-defined sequence; and
selecting families and subfamilies related to statistical models achieving high scores respective of the user-defined sequence.

16. The method of claim 11, further comprising communicating contents of the ontology to the user in accordance with user navigation selections.

17. The method of claim 11, further comprising:
receiving user defined text; and
selecting functional categories and subcategories by matching the text to contents of the ontology.

18. The method of claim 11, further comprising:
receiving a user-defined sequence; and
selecting functional categories and subcategories related to statistical models achieving high scores respective of the user-defined sequence.

19. The method of claim 11, further comprising:
receiving a set of database selections from the user; and
identifying sequences in a subset of multiple sequence datastores based on the database selections.

20. The method of claim 11, further comprising:
receiving a user selection of a Boolean operator; and
identifying the multiple alignments in accordance with the Boolean operator.

21. A method for constructing a browsable database for use with biological information, comprising:
clustering biological sequences into families based on global sequence similarity, wherein the biological sequences include at least one of protein sequences and gene sequences;
aligning the families by generating statistical models based on biological sequence clusters associated with the families; and
dividing the families into subfamilies of sequences sharing a common functional attribute, including at least one of molecular function and biological process.

22. The method of claim 21, further comprising extending an original family to include additional members based on the statistical models.

23. The method of claim 21, further comprising producing family trees based on the alignments.

24. The method of claim 21, further comprising selecting curators based on areas of expertise.

25. The method of claim 21, further comprising employing curators to review and annotate family trees in a distance tree context, wherein a curator links a distance tree of a family to sequence-level annotations related to sequences in the family.

26. The method of claim 21, further comprising providing subfamilies with biologically meaningful names.

27. The method of claim 21, further comprising assigning families and subfamilies to appropriate function and process categories of a biological function ontology.

28. The method of claim 21, further comprising scoring the statistical models against biological sequences.

29. The method of claim 21, further comprising relating biological sequences to functions associated with statistical models achieving high scores respective of those biological sequences.

30. The method of claim 21, further comprising training a statistical model on sequences related to a subfamily.
